(19) United States
(12) Patent Application Publication
(10) Pub. No.: US 2016/0298165 A1
(43) Pub. Date: Oct. 13, 2016

(54) METHOD OF TREATING TUBERCULOSIS

(71) Applicant: ALBERT EINSTEIN COLLEGE OF MEDICINE, INC,, Bronx, NY (US)

(72) Inventors: Travis Hartman, Bronx, NY (US); Michael Berney, Pelham, NY (US); William R Jacobs, JR., Pelham, NY (US); Catherine Vilheze, Flushing, NY (US)

(73) Assignee: ALBERT EINSTEIN COLLEGE OF MEDICINE, INC., Bronx, NY (US)

(21) Appl. No.: 14/785,361

(22) PCT Filed: Apr. 25, 2014
(86) PCT No.: PCT/US14/35448

§ 371 Date: Oct. 19, 2015

Related U.S. Application Data
(60) Provisional application No. 61/816,363, filed on Apr. 26, 2013.

(51) Int. Cl. C12Q 1/32; A61K 31/4409; A61K 45/06; G01N 33/569; A61K 31/133; A61K 31/7036; A61K 31/506; A61K 31/496; A61K 31/4965

(52) U.S. Cl. CPC C12Q 1/32; A61K 31/496; A61K 31/4409; A61K 45/06; A61K 31/4965; A61K 31/133; A61K 31/7036; A61K 31/506; G01N 33/5695; G01N 2333/35; G01N 2500/02

(57) ABSTRACT

The present invention addresses a need for improved methods of treating M. tuberculosis infection using enhancers of respiration, and compositions for treating tuberculosis as well as assays for identifying novel agents for treating M. tuberculosis infection.

### CROSS-REFERENCE TO RELATED APPLICATIONS

This application claims benefit of U.S. Provisional Application No. 61/816,363, filed Apr. 26, 2014, the contents of which are hereby incorporated by reference.

### STATEMENT OF GOVERNMENT SUPPORT

This invention was made with government support under grant numbers NIH AI090473, AI26170 and AI-051519 awarded by the National Institutes of Health. The government has certain rights in the invention.

### BACKGROUND OF THE INVENTION

The achievements of the World Health Organization's “Stop TB” program have been largely overlooked by the media, but mortality and incidence rates for clinically diagnosed tuberculosis have fallen for the second straight year (WHO, 2012a). Although the estimated 1.4 million deaths attributed to the disease are still unacceptably high for a curable disease, the success of the WHO program can be partially ascribed to the Directly Observed Therapy Short-course (DOTS) strategy, which closely monitors patient adherence to chemotherapeutic regimens. Strict adherence to treatment guidelines is thought to limit the risk of recurrence in individuals, thus limiting the spread of strains which have undergone a selection for resistance to a particular antibiotic (WHO, 2012a). TB3 relapse is a complicated phenomenon influenced by local public health conditions; for instance, countries with low burden may have increases relapse rates due to reactivation of latent TB (Jasmer et al., 2004), while relapses in areas in which TB is endemic may be largely due to reinfection (Narayanan et al., 2010). With clinical descriptions of multiple (MDR), extensively (XDR), and totally (TDR) drug resistant strains in the literature (Shah et al., 2011; Udwadia and Amale, 2012), TB is winning the war by accumulating resistance alleles before an effective chemotherapy can be developed. Patient compliance and cure rates may be improved if a regimen can be devised which cuts the duration of antibiotic treatment to a more manageable course.

The current recommended chemotherapy for treatment of susceptible pulmonary tuberculosis entails a two month course with daily administration of the front line antibiotics Isoniazid (INH), Rifampicin (RIF), Pyrazinamide (PZA), and Ethambutol (EMB); followed by a four month continuation phase of INH and RIF (WHO, 2012b). MDR and XDR strains require treatment which regularly lasts two years or more. If followed in its entirety, these programs are generally successful in reducing bacterial numbers in patients to below the limit of detection, effectively “curing” tuberculosis. The necessity for such a protracted regimen is thought to be due to the presence of persister cells which are insensitive to antibiotic treatment and only succumb to drug pressure at a reduced rate (Dhar and McKinney, 2007; Sacchettini et al., 2008). Importantly, persister cells are a fraction of the population that do not harbor genetically encoded features which render them permanently resistant; their survival mechanism is temporary, and sensitivity to antibiotics can be restored upon regrowth (Bigger, 1944; Keren et al., 2004; Wiuff et al., 2005). In no other organism does the presence of this persister population beget more dire consequences than in the context of Mycobacterium tuberculosis infection.

Bacterial persistence is thought to be a phenotypic phenomenon affecting a sub-population of cells in which replication rate is reduced. Landmark studies using time-lapse microscopy have shown that this slowly growing sup-population can originate in stationary phase or appear spontaneously during growth in mutant strains of E. coli (Balaban et al., 2004). Intriguingly, the proportion of persister cells as part of a population can be reduced by repeated serial passage through fresh media (Keren et al., 2004), suggesting that persistence is the phenotypic readout of a heterogeneity in metabolic control of growth. More recent work has sought genetic contributors to the persister phenotype; yet despite these efforts description of a coherent mechanism remains elusive (Lewis, 2010).

### SUMMARY OF THE INVENTION

Kinetics of multidrug killing of M. tuberculosis by the two front-line antibiotics Isoniazid and Rifampicin can be improved in a strain that harbors a deletion in one of the operons encoding putative succinate dehydrogenases. Stable isotope labeling and mass spectrometry were used to show that this enzyme functions as a succinate dehydrogenase during aerobic growth, and its deletion results in an increased proportion of reduced electron carrier in the membrane. The effect of this reinstatement of redox homeostasis is increased respiratory flux, resulting in oxygen consumption that continues unabated until oxygen is depleted. Results suggest that drugs which target SdhI or enhance respiration would shorten tuberculosis chemotherapy.

A method is provided for identifying an agent as an enhancer of an anti-tuberculosis medication, or as a combination agent for treating resistant and/or dormant Mycobacteria tuberculosis infection in a subject, comprising:
quantifying activity of an amount of Mycobacteria tuberculosis succinate dehydrogenase I under conditions permitting succinate dehydrogenase I activity,
contacting the Mycobacteria tuberculosis succinate dehydrogenase I with the agent and quantifying activity of the amount of Mycobacteria tuberculosis succinate dehydrogenase I in the presence of the agent, comparing the quantified amounts, and identifying the agent as an enhancer, or not, of an anti-tuberculosis medication, or as a combination agent, or not, for treating resistant and/or dormant Mycobacteria tuberculosis infection, wherein a decreased activity of Mycobacteria tuberculosis succinate dehydrogenase I in the presence of the agent as compared to in the absence of the agent indicates that the agent is an enhancer of an anti-tuberculosis medication, or is a combination agent for treating resistant and/or dormant Mycobacteria tuberculosis infection.

A method is provided for identifying an agent as an enhancer of an anti-tuberculosis medication, or as a combination agent for treating resistant and/or dormant Mycobacteria tuberculosis infection in a subject, comprising: quantifying respiration of a Mycobacteria tuberculosis, contacting the Mycobacteria tuberculosis with the agent and quantifying respiration of Mycobacteria tuberculosis in the presence of the agent, comparing the quantified amounts, and identifying the agent as an enhancer, or not, of an anti-tuberculosis medication, or as a combination agent, or not, for treating resistant and/or dormant Mycobacteria tuberculosis infection, wherein an increased respiration rate of Mycobacteria tuberculosis in the presence of the agent as compared to in the absence of the agent indicates that the agent is an enhancer of an anti-tuberculosis medication, or is a combination agent for treating resistant and/or dormant Mycobacteria tuberculosis infection.

Also provided is a method of enhancing efficacy of an anti-tuberculosis medication in treating tuberculosis in a subject or in treating resistant and/or dormant Mycobacteria tuberculosis infection in a subject, comprising: administering to the subject who has, is or will be receiving the anti-tuberculosis medication an amount of an enhancer of Mycobacteria tuberculosis respiration effective to enhance the efficacy of an anti-tuberculosis medication in treating tuberculosis or resistant and/or dormant Mycobacteria tuberculosis infection.

Also provided is a method of treating tuberculosis in a subject, or of treating resistant and/or dormant Mycobacteria tuberculosis infection in a subject, comprising administering to the subject an amount of an anti-tuberculosis antibiotic medication and an amount of an enhancer of Mycobacteria tuberculosis respiration effective to treat tuberculosis in a subject, or effective to treat resistant and/or dormant Mycobacteria tuberculosis infection in a subject.

Also provided is a composition comprising an anti-tuberculosis medication and an enhancer of Mycobacteria tuberculosis respiration.

### BRIEF DESCRIPTION OF THE DRAWINGS

FIGS. 1A-1B: Increased susceptibility of M. tuberculosis strains mc²6230Δsdh1 and mc²6230Δsdh2 to drug treatments as compared with strain mc²6230. FIG. 1B is a schematic diagram showing the genes present in the mc²6230Δsdh1 and mc²62304Δsdh2 strains.

FIG. 2: Schematic diagram showing the metabolism of different M. tuberculosis strains under aerobic and hypoxic conditions. Levels of each metabolic intermediate are shown for each strain.

FIGS. 3A-3B: Schematic diagram showing the results of ¹³C a radiolabeling experiment tracking the progress of carbon through the metabolism of M. tuberculosis strain mc²6230Δsdh1. FIG. 3B is a schematic diagram showing the results of ¹³C a radiolabeling experiment tracking the progress of carbon through the metabolism of M. tuberculosis strain mc²6230Δsdh2.

FIGS. 4A-4B: Differing survival times for various strains of M. tuberculosis under ordinary conditions and under treatment with carbonyl cyanide 3-chlorophenylhydrazone (CCCP). FIG. 4B shows the ratio of reduced MK-9 (MK-9_{red}) to oxidized MK-9 (MK-9 (MK-9_{oxid}) in each strain of M. tuberculosis.

FIGS. 5A-5E: Duration of cellular respiration for each strain of M. tuberculosis. FIG. 5B shows the growth of M. tuberculosis in the presence of glycerol. FIG. 5C shows the growth of M. tuberculosis in the presence of 20 mM glucose. FIG. 5D shows the growth of M. tuberculosis in the presence of 10 mM succinate. FIG. 5E shows the growth of M. tuberculosis in the presence of 800 μM caproic acid.

FIGS. 6A-6C: Survival rates of several strains of M. tuberculosis two days after treatment with drugs. FIG. 6B shows the survival rates of several strains of M. tuberculosis five days after treatment with drugs. FIG. 6C shows survival rates of several strains of M. tuberculosis in hypoxic conditions after treatment with rifampicin.

FIG. 7: Growth rates, via optical density, of several mutant strains of M. tuberculosis.

FIG. 8: Respiration rates, via optical density, of several sdh mutants of M. tuberculosis.

FIG. 9 : Oxygen consumption by several mutant strains of M. tuberculosis.

FIG. 10: Change in pH of the cultures batches for several mutant strains of M. tuberculosis.

FIG. 11: Growth of M. tuberculosis under hypoxic conditions.

FIG. 12: Growth of several strains of M. tuberculosis under various conditions,

FIG. 13: Growth of several strains of M. tuberculosis under various conditions.

FIG. 14A-C. Viability of M. tuberculosis grown at 37° C. with shaking treated with: A. INH (7.3 μM), cysteine (4 mM); B. RIF (1.2 μM), cysteine (4 mM); C. EMB (0.5 mM), cysteine (4 mM). The compound combination used the same concentration of compounds as the individual treatments. D. M. tuberculosis H37Rv rpoB H445R, a RIF-resistant strain, was treated with RIF, INH, cysteine or a combination at the same concentration as for A, B, or C. Aliquots were taken at indicated time and plated to determine CFUs. The curves represent the average of at least 3 independent experiments with error bars indicating standard deviation.

FIG. 15A-D. The combination INH/Cysteine and RIF/Cysteine increases ROS production and DNA damage. A. M. tuberculosis H37Rv ΔRD1 ΔpanCD was treated with INH (7.3 μM), cysteine (4 mM), or a combination using the same concentrations as the individual treatments, and then stained with dihydroethidium. ROS levels were measured by flow cytometry for up to 7 days and compared to the untreated samples. Data show mean value with standard deviation (n=3), B. M. tuberculosis H37Rv ΔRD1 ΔpanCD was treated with RIF (1.2 μM), cysteine (4 mM), or a combination using the same concentrations as the individual treatments, and analyzed as in A. Data show mean value with standard deviation (n=3). C. The samples in A and B were also used to determine the % of double stranded DNA break. The experiments were done in triplicate and the average with standard deviation is plotted. D. M. tuberculosis H37Rv ΔRD1 ΔpanCD was treated with RIF (1.2 μM), cysteine (4 mM), and a combination under anaerobic conditions (<0.0001% O₂, 5% CO₂, 10% H₂, 85% N₂). Aliquots were taken at indicated time and plated to determine CFUs. Average with standard deviation is shown (n=2).

FIG. 16A-B The combined effect of cysteine with INH or RIF is iron-dependent. A. M. tuberculosis H37Rv ΔRD1 ΔpanCD was treated with INH (7.3 μM) and cysteine (4 mM) (left panel) or RIF (1.2 μM) and cysteine (4 mM) (right panel) for 3 days at 37° C. The combination used the same concentrations as the individual treatments. Free ferrous ions were extracted and measured as described in the experimental section. Average with standard deviation is shown (n=3). B. M. tuberculosis H37Rv was treated with INH (7.3 μM), cysteine (4 mM), DFO (152 μM), (left panel) or RIF (1.2 μM), cysteine (4 mM), DFO (152 μM) (right panel) for 21 days at 37° C. The combination used the same concentrations as the individual treatments. Aliquots were taken at the indicated time and plated for CPUs. Average with standard deviation is shown (n=2).

FIG. 17A-B. High levels of cystine and not cysteine are found in M. tuberculosis treated with cysteine, INH/Cyst, and RIF/Cyst. A. UPLC-MS determination of the relative amount of cystine compared to untreated in If tuberculosis H37Rv ΔRD1 ΔpanCD treated with INH (7.3 μM), RIF (1.2 μM), cysteine (4 mM), or a combination using the same concentrations as the individual treatments for 24 h. Average with standard deviation is plotted (n=3). B. Total thiol concentrations were measured from extracts of M. tuberculosis H37Rv ΔRD1 ΔpanCD treated as above. Thiol concentrations were normalized to protein concentration. Average with standard deviation is plotted (n=3).

FIG. 18A-B: VC acts as a pro-oxidant in M. tuberculosis. (A) M. tuberculosis mc26230 was grown under aerobic conditions and then shifted to an anaerobic chamber (o0.0001% O2, 5% CO₂, 10% H₂ and 85% N₂). After 24 h, the culture was diluted 1/20 with hypoxic media and treated with 4 mM VC. Aliquots were taken at indicated time and plated to determine CPUs. Plates were incubated in an aerobic incubator. (B) The mycothiol-deficient strain H37Rv DmshA, grown in Middlebrook 7H9 supplemented with OADC instead of ADS, was treated with 4 mM VC. Growth was followed by plating for CFUs at different times. Both experiments were done in duplicate and the average with s.d. is plotted.

### DETAILED DESCRIPTION OF THE INVENTION

Herein it is disclosed that deletion of a central metabolic gene in M. tuberculosis removes the metabolic resistance to rapid bactericidal activity by INH and RIF. The deletion of a gene, which is demonstrate by metabolomic analyses to encode to encode succinate dehydrogenase activity, mediates this enhanced rate of cell death in the presence of and RIF. Moreover, this mutant has lost its ability to stop respiration in tow oxygen conditions. It is postulated that the entry to persistence requires this biochemical ability to regulate respiration, providing novel avenues to develop therapeutic strategies to shorten TB chemotherapy.

A method is provided for identifying an agent as an enhancer of an anti-tuberculosis medication, or as a combination agent for treating resistant and/or dormant Mycobacteria tuberculosis infection in a subject, comprising:
quantifying activity of an amount of Mycobacteria tuberculosis succinate dehydrogenase I under conditions permitting succinate dehydrogenase I activity, contacting the Mycobacteria tuberculosis succinate dehydrogenase I with the agent and quantifying activity of the amount of Mycobacteria tuberculosis succinate dehydrogenase I in the presence of the agent, comparing the quantified amounts, and identifying the agent as an enhancer, or not, of an anti-tuberculosis medication, or as a combination agent, or not, for treating resistant and/or dormant Mycobacteria tuberculosis infection, wherein a decreased activity of Mycobacteria tuberculosis succinate dehydrogenase in the presence of the agent as compared to in the absence of the agent indicates that the agent is an enhancer of an anti-tuberculosis medication, or is a combination agent for treating resistant and/or dormant Mycobacteria tuberculosis infection.

In an embodiment, increased activity of Mycobacteria tuberculosis succinate dehydrogenase I in the presence of the agent as compared to in the absence of the agent indicates that the agent is not an enhancer of an anti-tuberculosis medication, or is not a combination agent for treating resistant and/or dormant Mycobacteria tuberculosis infection.

Also provided is a method for identifying an agent as an enhancer of an anti-tuberculosis medication, or as a combination agent for treating resistant and/or dormant Mycobacteria tuberculosis infection in a subject, comprising: quantifying respiration of Mycobacteria tuberculosis, contacting the Mycobacteria tuberculosis with the agent and quantifying respiration of Mycobacteria tuberculosis in the presence of the agent, comparing the quantified amounts, and identifying the agent as an enhancer, or not, of an anti-tuberculosis medication, or as a combination agent, or not, for treating resistant and/or dormant Mycobacteria tuberculosis infection, wherein an increased respiration rate of Mycobacteria tuberculosis in the presence of the agent as compared to in the absence of the agent indicates that the agent is an enhancer of an anti-tuberculosis medication, or is a combination agent for treating resistant and/or dormant Mycobacteria tuberculosis infection.

In an embodiment, an increased respiration rate of Mycobacteria tuberculosis in the presence of the agent as compared to in the absence of the agent indicates that the agent is not an enhancer of an anti-tuberculosis medication, or is not a combination agent for treating resistant and/or dormant Mycobacteria tuberculosis infection.

The increased respiration rate of Mycobacteria tuberculosis may be measured by direct or indirect means known in the art. In an embodiment, the Mycobacteria tuberculosis are maintained in a fixed amount of O₂. In a further embodiment, the rate or O₂ consumption of the Mycobacteria tuberculosis maintained in a fixed amount of O₂ is quantified as a measure of respiration. In an embodiment, the growth rate of the Mycobacteria tuberculosis under one or more O₂ tensions is determined.

In an embodiment of the methods described herein, the agent or enhancer of respiration is an inhibitor of Mycobacteria tuberculosis succinate dehydrogenase I. In an embodiment of the methods described herein, the agent or enhancer of respiration is a small organic molecule of 2000 daltons or less, an aptamer, an RNAi molecule, a peptide, a fusion protein, an antibody, or a fragment of an antibody. In an embodiment of the methods described herein, the enhancer of respiration inhibits expression of Mycobacteria tuberculosis succinate dehydrogenase I or inhibits activity of Mycobacteria tuberculosis succinate dehydrogenase I. In an embodiment of the methods described herein, the Mycobacteria tuberculosis succinate dehydrogenase I is encoded by an operon comprising Mycobacteria tuberculosis genes Rv0247c, Rv0248c, and Rv0249c. In an embodiment of the methods described herein, the Mycobacteria tuberculosis are maintained in a bioreactor system in which one or more of O₂ tension, optical density, redox midpoint potential and pH are measured simultaneously. In an embodiment of the methods described herein, a growth curve of the Mycobacteria tuberculosis is quantified as oxygen is depleted by the Mycobacteria tuberculosis.

In an embodiment, the Mycobacteria tuberculosis succinate dehydrogenase I inhibitor binds in the succinate pocket of the succinate dehydrogenase I. In an embodiment, the Mycobacteria tuberculosis succinate dehydrogenase I inhibitor binds in the ubiquinone pocket of the succinate dehydrogenase I. Ubiquinone type inhibitors include carboxin and thenoyltrifluoroacetone. Succinate-analogue inhibitors include the synthetic compound malonate as well as the TCA cycle intermediates, malate and oxaloacetate.

In an embodiment, the Mycobacteria tuberculosis is an H37Rv strain M. tuberculosis.

In an embodiment of the inventions described herein, the Mycobacterium tuberculosis is one of the following: Mycobacterium tuberculosis H37Rv, BTB05-552, BTB05-559, CDC1551, CTRI-2, F11, H37, H37Ra, HN878, KZN 1435, KZN 4207, KZN R506, KZN V2475, R1207, RGTB327, S96-129, X122, ‘98-R604 INH-RIF-EM’, 02_1987, 210, 94_M4241A, C, CDC1551A, CPHL_A, CTRI-4, EAS054, GM 1503, K85, KZN 605, OSDD071, OSDD504, OSDD518, SUMu001, SUMu002, SUMu003, SUMu004, SUMu005, SUMu006, SUMu007, SUMu008, SUMu009, SUMu010, SUMu011, SUMu012, T17, T46, T85, T92, W-148, str. Haarlem, 210_16C10, 210_16C2_24C1, 210_16C2_24C2, 210_32C4, 210_4C15, 210_4C15_16C1, 210_4C15_16C1_48C1, 210_4C15_16C1_48C2, 210_4C15_16C1_56C1, 210_4C15_16C1_56C2, 210_4C31, 210_4C31_16C1, 210_4C31_16C1_24C1, 210_4C31_16C1_40C1, 210_4C31_16C2, 210_8C1, 210_8C6, BC, CTRI-3, H37Rv_2009. NJT210GTG, str. Erdman=ATCC 35801, str. Erdman WHO, CCDC5079, CCDC5180, RGTB423, UT205, CTRI-1, H37RvAE, H37RvCO, H37RvHA, H37RvJO, H37RvLP, H37RvMA, LAM7, NCGM2209, RGTB306, WX1, WX3, XDR1219, XDR1221, str. Beijing/W BT1, or str. Erdman (ATCC 35801).

As used herein a “combination agent” for treating resistant and/or dormant Mycobacteria tuberculosis infection in a subject is an agent that is to be used as an adjuvant for, or with, an anti-tuberculosis agent. An anti-tuberculosis agents are well-known in the art.

This invention further provides a method of enhancing efficacy of an anti-tuberculosis medication in treating tuberculosis in a subject or in treating resistant and/or dormant Mycobacteria tuberculosis infection in a subject, comprising:
administering to the subject who has, is or will be receiving the anti-tuberculosis medication an amount of an enhancer of Mycobacteria tuberculosis respiration effective to enhance the efficacy of an anti-tuberculosis medication in treating tuberculosis or resistant and/or dormant Mycobacteria tuberculosis infection.

This invention also provides a method of treating tuberculosis in a subject, or of treating resistant and/or dormant Mycobacteria tuberculosis infection in a subject, comprising administering to the subject an amount of an anti-tuberculosis antibiotic medication and an amount of an enhancer of Mycobacteria tuberculosis respiration effective to treat tuberculosis in a subject, or effective to treat resistant and/or dormant Mycobacteria tuberculosis infection in a subject.

In an embodiment of the methods, the agent or the enhancer of respiration comprises a free thiol group or is vitamin C. In an embodiment, the agent or the enhancer of respiration comprises a free thiol group and is a cysteine, dithiothreitol or penicillamine.

In an embodiment, the anti-tuberculosis medication comprises isoniazid. In an embodiment, the anti-tuberculosis medication comprises rifampicin. In an embodiment, tuberculosis is multidrug-resistant (MDR). In an embodiment, the tuberculosis is extensively drug-resistant (XDR). In an embodiment, the MDR is resistant to an anti-tuberculosis medication other than the anti-tuberculosis medication being used in the method to treat the TB. For example, the MDR is rifampicin-resistant and the method comprises treating the MDR TB with an enhancer of respiration and isoniazid. For example, the MDR is isoniazid-resistant and the method comprises treating the MDR TB with an enhancer of respiration and rifampicin.

In an embodiment, the enhancer of respiration is cysteine. In an embodiment, the amount of cysteine and anti-tuberculosis medication combined is sufficient to kill Mycobacteria tuberculosis. In an embodiment, the amount of cysteine and anti-tuberculosis medication combined enhances the efficacy of x in killing Mycobacteria tuberculosis.

In an embodiment, the enhancer of respiration comprises D-cysteine. In an embodiment, the enhancer of respiration comprises L-cysteine.

In an embodiment subject the amount of an anti-tuberculosis antibiotic medication and amount of an enhancer of Mycobacteria tuberculosis respiration are administered in a single composition.

In an embodiment, the enhancer of Mycobacteria tuberculosis respiration compound comprises a free thiol group, but the compound is other than N-acetylcysteine. In an embodiment, the enhancer of Mycobacteria tuberculosis respiration compound comprises two free thiol groups. In an embodiment, the enhancer of Mycobacteria tuberculosis respiration compound comprises more than two free thiol groups.

In an embodiment of the methods described herein, the anti-tuberculosis medication comprises one or more of isoniazid, rifampicin, pyrazinamide, and ethambutol. In an embodiment of the methods described herein, the anti-tuberculosis medication comprises rifampicin and isoniazid.

In an embodiment, the anti-tuberculosis medication comprises one or more of isoniazid, rifampicin, pyrazinamide, ethambutol, a fluoroquinolone, amikacin, capreomycin or kanamycin. Exemplary fluoroquinolones include ofloxacin and moxifloxacin. In an embodiment, the anti-tuberculosis medication comprises rifampicin and isoniazid.

Also provided is a composition comprising an anti-tuberculosis medication and an enhancer of Mycobacteria tuberculosis respiration.

In an embodiment, the composition comprises a pharmaceutically acceptable carrier. In an embodiment of the composition, the anti-tuberculosis medication comprises one or more of isoniazid, rifampicin, pyrazinamide, and ethambutol. In an embodiment of the composition, the enhancer of respiration inhibits expression of Mycobacteria tuberculosis succinate dehydrogenase I or inhibits activity of Mycobacteria tuberculosis succinate dehydrogenase I.

In an embodiment of the composition, the anti-tuberculosis medication comprises one or more of isoniazid, rifampicin, pyrazinamide, and ethambutol.

In an embodiment of the composition, the enhancer of respiration inhibits expression of Mycobacteria tuberculosis succinate dehydrogenase I or inhibits activity of Mycobacteria tuberculosis succinate dehydrogenase I.

In an embodiment of the composition, the enhancer of respiration comprises a free thiol or vitamin C. In an embodiment, the enhancer of respiration comprises a free thiol. In an embodiment, the enhancer of respiration comprises a free thiol in a small organic molecule of less than 2,000 daltons. In an embodiment, the enhancer of respiration comprises vitamin C.

In an embodiment of the composition, the enhancer of respiration comprises a free thiol and is a cysteine, dithiothreitol or penicillamine. In an embodiment of the composition, the enhancer of respiration comprises vitamin C.

In an embodiment of the composition, anti-tuberculosis medication comprises isoniazid. In an embodiment of the composition, the anti-tuberculosis medication comprises rifampicin. In an embodiment, the amount of the anti-tuberculosis and the enhancer of respiration are sufficient to kill Mycobacteria tuberculosis. In an embodiment, the amount of the enhancer of respiration is sufficient to enhance the amount of the anti-tuberculosis in killing Mycobacteria tuberculosis.

In an embodiment or the methods and compositions described herein, the enhancer of respiration is an enhancer of Mycobacteria tuberculosis respiration. In an embodiment, “respiration” as recited herein shall mean aerobic respiration.

The methods disclosed herein involving subjects can be used with any species capable of being infected by mycobacteria, preferably M. Tuberculosis. In a preferred embodiment, the subject is a mammalian subject. Most preferably, the mammal is a human.

### EXPERIMENTAL RESULTS

### Example 1

TraSH Screen reveals the importance of electron transport in kinetics of antibiotic induced mycobacterial death: Progress toward the goal of shortening TB chemotherapy requires knowledge of the physiological state of drug tolerant bacilli. Although inhibition of mycolic acid. biosynthesis is apparent within hours after the addition of INH, and RIF uptake occurs within minutes (Winder and Collins, 1970; Piddock, 2000); it is still not clear why a fraction of M. tuberculosis cells are not sterilized after eight weeks under these conditions in vivo (Wallis et al., 1999). In an effort to uncover the mechanisms of tuberculosis persistence, a Transposon Site Hybridization (TraSH) screen (Sassetti et al., 2001) was performed for mutants unable to survive a short course combination treatment of the front line drugs INH and RIF in vitro. The screen was performed on M. tuberculosis strain mc²7000 (Sambandamurthy et al., 2002); after four days of treatment in an in vitro tolerance model, genomic DNA was isolated and evaluated for insertions which were underrepresented with respect to untreated cells (Table 2).

TABLE 2
M. tuberculosis strains used in this work.

| strain number | parent | genotype | modification |
|---|---|---|---|
| mc²2 | — | RD1⁺ panCD⁺ | H37Rv |
| mc²7000 | mc²2 | ΔRD1 ΔpanCD | |
| mc²6230 | mc²2 | ΔRD1 ΔpanCD | |
| mc²7292 | mc²2 | RD1⁺ panCD⁺ | Δ(Rv0247c, Rv0248c, Rv0249c) |
| mc²7293 | mc²2 | RD1⁺ panCD⁺ | Δ(sdhC, sdhD, sdhA, sdhB) |
| mc²7294 | mc²7292 | RD1⁺ panCD⁺ | Δ(Rv0247c, Rv0248c, Rv0249c) :: Pₕₛₚ₆₀(Rv0247c, Rv0248c, |
| | | | Rv0249c) |
| mc²7295 | mc²7293 | RD1⁺ panCD⁺ | Δ(sdhC, sdhD, sdhA, sdhB) :: Pₕₛₚ₆₀(sdhC, sdhD, sdhA, sdhB) |
| mc²7296 | mc²6230 | ΔRD1 ΔpanCD | Δ(Rv0247c, Rv0248c, Rv0249c) |
| mc²7297 | mc²6230 | ΔRD1 ΔpanCD | Δ(sdhC, sdhD, sdhA, sdhB) |
| mc²7298 | mc²7296 | ΔRD1 ΔpanCD | Δ(Rv0247c, Rv0248c, Rv0249c) :: Pₕₛₚ₆₀(Rv0247c, Rv0248c, |
| | | | Rv0249c) |
| mc²7299 | mc²7297 | ΔRD1 ΔpanCD | Δ(sdhC, sdhD, sdhA, sdhB) :: Pₕₛₚ₆₀(sdhC, sdhD, sdhA, sdhB) |
| mc²7300 | mc²7296 | ΔRD1 ΔpanCD | Δ(Rv0247c, Rv0248c, Rv0249c) :: Pₜₑₜ₂(Rv0247c, Rv0248c, |
| | | | Rv0249c) |
| mc²7301 | mc²7297 | ΔRD1 ΔpanCD | Δ(sdhC, sdhD, sdhA, sdhB) Pₜₑₜ₂:: (sdhC, sdhD, sdhA, sdhB) |
| mc²5871 | mc²6230 | ΔRD1 ΔpanCD | Δndh |
| mc²5872 | mc²6230 | ΔRD1 ΔpanCD | Δndh pMV361::ndh |
| mc²5873 | mc²6230 | ΔRD1 ΔpanCD | ΔndhA |
| mc²5874 | mc²6230 | ΔRD1 ΔpanCD | ΔndhA pMV361::ndhA |

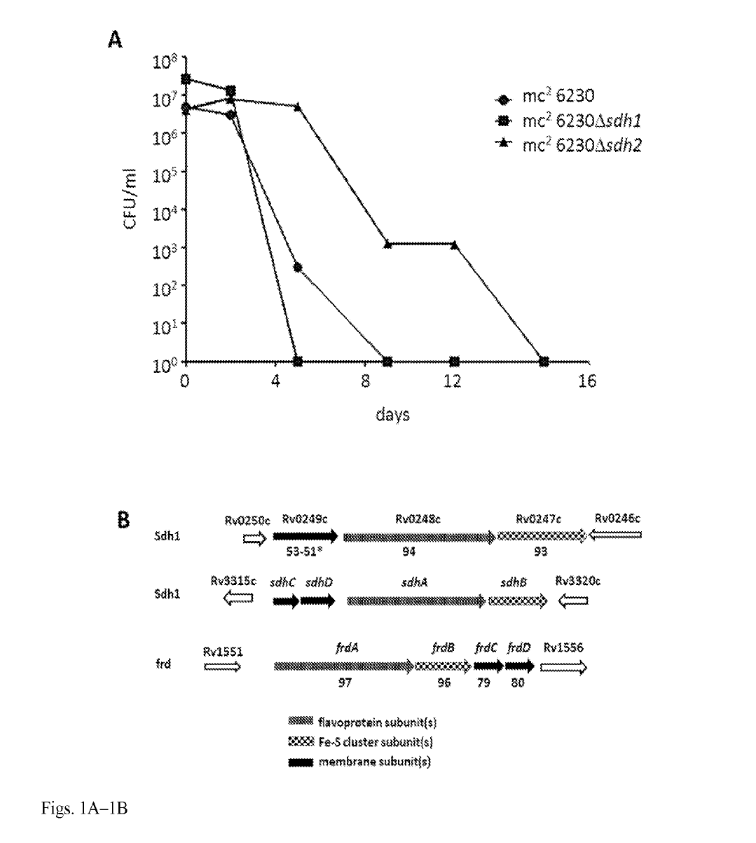

Functional classification of hits revealed that electron transport and redox control were important processes in kinetics of antibiotic killing. In addition to proteins of unknown function, a class of genes thought to affect cell wall permeability was noted and will be discussed in future work. Specialized transduction (Bardarov et al., 2002) was performed to obtain precise deletions of individual genes for each TraSH hit as well as deletion of relevant operons (not shown), and these strains were retested with INH+RIF treatment at 10 times the MIC. Based on the results of this verification treatment, members of an operon encoding a putative succinate dehydrogenase (Rv0247c, Rv0248c, Rv0249c), which was consistently more susceptible to the drug combination (FIG. 1A), were chosen for further study. Mutant strains were complemented using integrated plasmids containing constitutive or inducible promoters (Supplemental Data). It is hypothesized that the kinetics of multi-drug killing is influenced by flux through the electron transport chain.

The putative operon Rv0247c through Rv0249c encodes a succinate dehydrogenase: Coupling of oxidative flux through the TCA cycle to the electron transport chain is accomplished by succinate dehydrogenase. M. tuberculosis has two putative succinate:quinone oxidoreductases as well as a putative quinol:fumarate oxidoreductase, they can be annotated as sdh1 and sdh2 (Berney and Cook, 2010), and frd, respectively. These enzymes are predicted to catalyze the oxidation of succinate to fumarate with a corresponding reduction of quinone to quinol, but physiologically, the succinate oxidation:fumarate reduction catalytic ratios are dependent on substrate concentrations and a critical redox potential (Cecchini et al., 2002; Lemos et al., 2002). Absolute pool sizes of metabolic intermediates are highly dynamic in living cells as a function of growth stage, pH, air mixture, and temperature, and the predominant direction of catalysis for each enzyme at any time cannot be inferred by annotation alone (FIG. 1B).

The sdh1 of M. tuberculosis was predicted to be essential for survival in mouse lung, while sdh2 was not (Sassetti and Rubin, 2003), and sdh1 also appears to be essential for the transition from slow to fast growth in a continuous culture system (Beste et al., 2009). Deletion of the annotated fumarate reductase, which was observed to be strongly upregulated in hypoxia, was recently described (Watanabe et al., 2011) but no obvious phenotype was observed in cells containing a null deletion of the operon. While constructing the precise deletions of the sdh genes (Table 2), it was discovered that there was a stationary phase exit defect in which sdh1 mutants were unable to be rescued from two month old cultures; sdh2 mutants grew poorly after a similar period (FIG. 7) while wt cultures exhibited no comparable difficulty surviving even eight months of stationary phase, suggesting these operons play a role in maintenance bioenergetics. Gene function was evaluated in a physiologically relevant context using a targeted metabolomic approach. Initial experiments compared the metabolome of H37Rv wild type and sdh mutants during tog phase growth and stationary phase adaptation, with similar analyses for mc²6230. These studies revealed similar trends in central carbon metabolism between the attenuated and virulent strains, suggesting that the attenuation had no appreciable effect on proportions of principal metabolic intermediates under standard culture conditions.

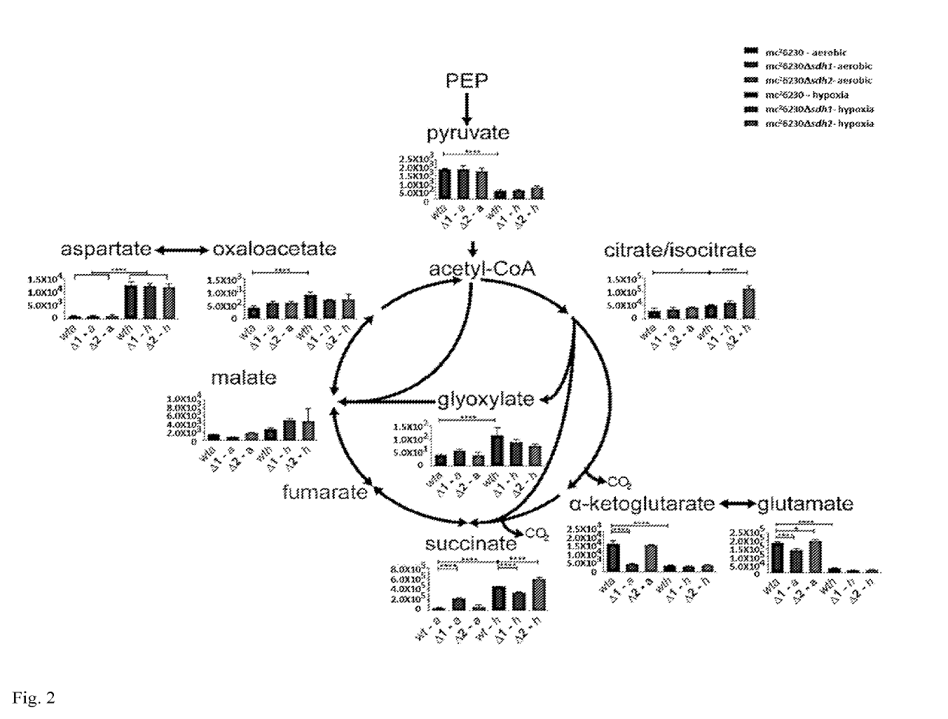

Next, qualitative differences in pool sizes of central carbon metabolites were sought using a targeted metabolomics approach for cells in aerobic growth and in an anaerobic model (Baughn et al., 2009) in which differences in activities of the enzymes may be revealed. Comparison of the mutant strains to parental mc26230 during log phase growth (in which oxidative flux of the TCA cycle predominates) revealed a 3.9-fold increase in intracellular succinate in the Δsdh1 strain and a 1.38-fold increase in the Δsdh2 strain. This was accompanied by a decrease of malate concentration in Δsdh1 to half that of the parental strain, whereas the Δsdh2 strain showed a 1.32-fold increase; these data indicate decreased succinate dehydrogenase activity in these strains (FIG. 2). The total intracellular succinate concentration of M. tuberculosis increases after 12 days of hypoxia (seven-fold in this system), white the concentration in the Δsdh1 mutant barely doubles. Conversely, total malate concentration rises slightly in the wt strain (1.7-fold), while the Δsdh1 mutant shows a seven-fold increase. Using this data, it is impossible to determine the exact source of the accumulation of intracellular succinate, but since total concentrations of α-ketoglutarate decrease, and glyoxylate, oxaloacetate and malate increase in hypoxia, a portion of the succinate is likely to be from the reported reductive flux of the TCA cycle. Consistent with reductive flux, during hypoxia similar changes in succinate concentrations were observed in the mutants relative to the wt (0.89-fold for Δsdh1 and 1.28-fold for Δsdh2) and malate concentrations were 2.16 and 1.75-fold increased, respectively. These data support decreased succinate dehydrogenase activity in the mutants during aerobic growth, particularly for the Δsdh1 strain; but do not conclusively show either Sdh to function as a fumarate reductase under hypoxia.

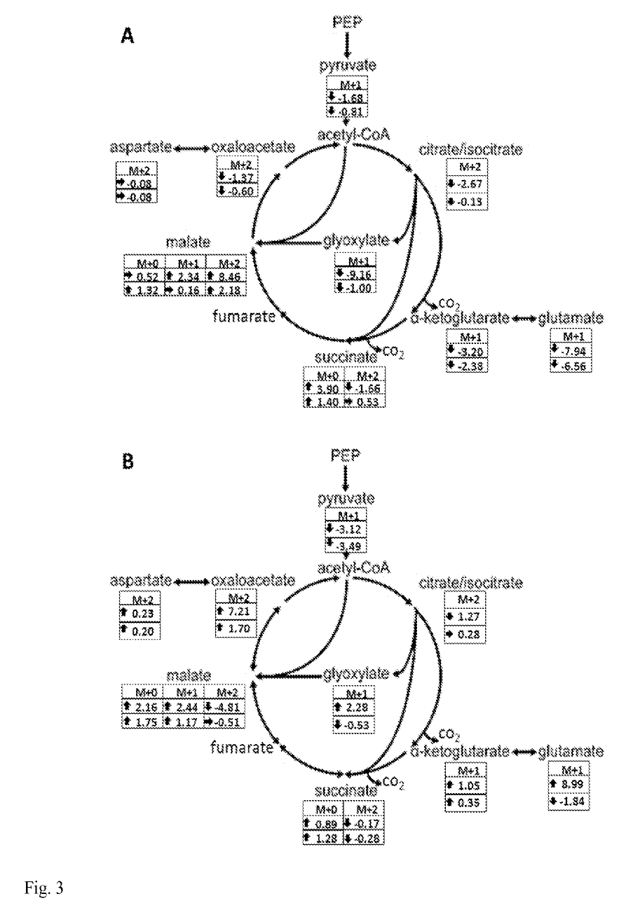

Based on the differences in metabolite pools, analyses were performed for the predominant direction of catalysis in the same hypoxia model using stable isotope labeling to determine flux. Cells were grown in complete media and labeled with [1.4-13C] aspartate in both aerobic tog phase growth and after twelve days in hypoxia using methods similar to those previously described (Watanabe et al., 2011). The fate of isotopically labeled carbon was traced in TCA intermediates (FIG. 3) and determined the proportion of each labeled metabolite with respect to all isotopologues for each intermediate. It is important to note that the proportion of labeled intermediates using this method—even in hypoxia—is less than 10%, so this study considered the labeled fraction of these metabolites and compared the proportion of each isotopologue with respect to the proportion of the same molecule in the parental strain for each condition.

During log phase growth the experiment produced a four-fold more unlabeled succinate in the Δsdh1 strain than the wild type, the proportion bearing two ¹³C atoms was decreased 1.66-fold with respect to the parental strain, whereas the proportion of malate containing 13C2 was increased over eight-fold (FIG. 3A). Because the increased intracellular succinate could be due to compensatory activity by the glyoxylate shunt which forms glyoxylate and succinate from isocitrate (McKinney et al., 2000), the incorporation of label into glyoxylate was examined; labeled glyoxylate indicated a nine-fold proportional decrease in ¹³C1 mass, suggesting a relatively small contribution from this pathway during aerobiosis. Additionally, the decreased total malate in the Δsdh1 mutant was similar to the parental strain in proportion of unlabeled malate, but there was an eight-fold increase in the M+2 isotopomer. It is possible that decreased activity of Mez (the malate oxidoreductase) might be responsible for the buildup of malate and indeed, a 1.68-fold decrease in M+1 pyruvate was detected in this mutant. These isotope ratios are consistent with a loss of succinate dehydrogenase activity in this mutant. The Δsdh2 strain had similar total succinate levels as the parent, and there was a 1.4-fold increase in unlabeled succinate and a 1.3-fold increase in unlabeled malate, supporting intact succinate dehydrogenase activity. In hypoxia, the proportion of M+2 malate was decreased in the Δsdh1 mutant 4.8-fold but the M+2 succinate was only slightly decreased (FIG. 3B), suggestive of intact or slightly increased Frd activity. The unlabeled succinate concentration (indicative of oxidative flux) was increased 0.89-fold, and M+0 malate was increased over two-fold in this strain. Conversely, the Δsdh2 mutant continued to show an increase in unlabeled succinate (0.53-fold) and a 1.32-fold increase in the proportion of unlabeled malate, indicative of intact Sdh activity (presumably) by sdh1.

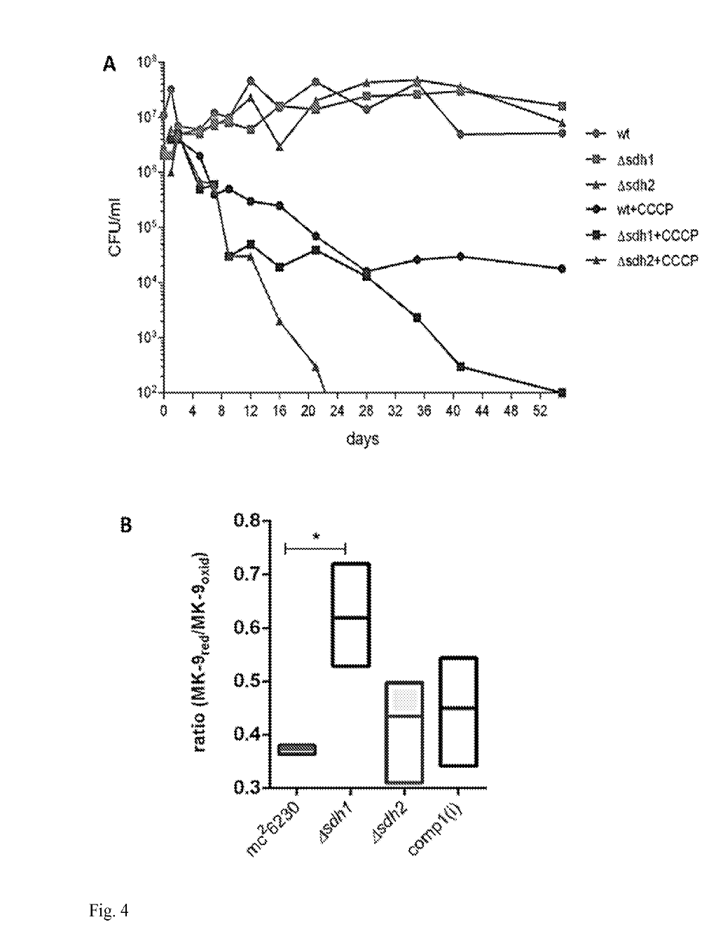

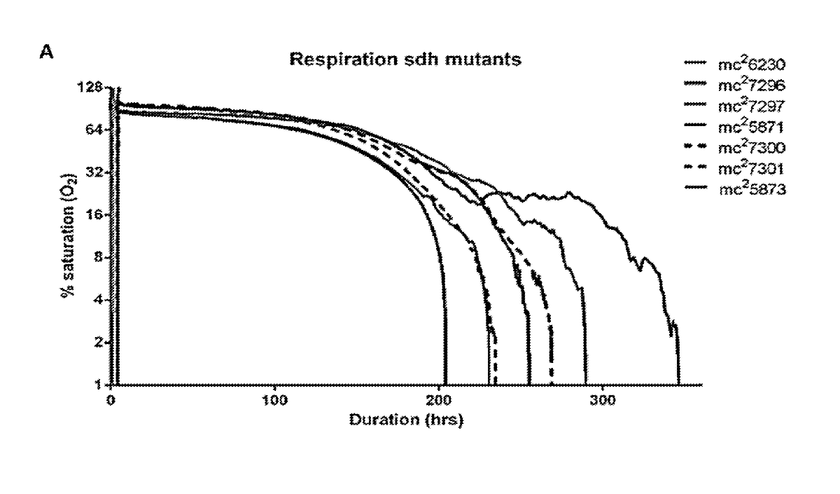

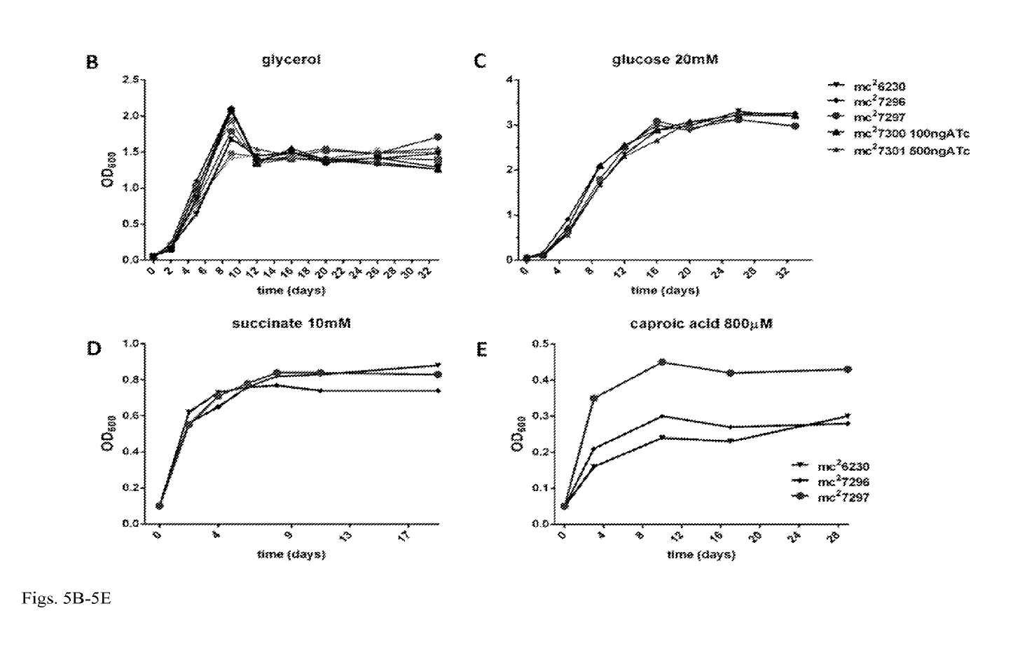

If it is true that sdh1 functions primarily oxidatively and sdh2 primarily reductively, one would expect to see differences in the ability of these mutants to survive disruption of the proton gradient in hypoxic conditions where Frd activity would contribute heavily to survival and Sdh activity would be less important, reflecting diminished need for anabolic metabolism. (FIG. 4A) shows survival in hypoxia after addition of the proton ionophore carbonyl cyanide 3-chlorophenylhydrazone (CCCP). Addition of CCCP to hypoxic cultures at a concentration below that required for complete dissipation of proton motive force (50-80 μM) allowed 0.1% of the parental inoculum to remain viable over the course of 56 days, while sdh2 was unable to be rescued after 22-24 days and no viable cells remained of the sdh1 mutant by the end of the experiment.

It is concluded from the metabolomic data, and corroborating phenotype of these mutants in hypoxia, that a functional reassignment should be considered for the operon encoded by Rv0247c-Rv0249c. This operon likely encodes the primary succinate dehydrogenase of M. tuberculosis and the operon encoded by sdhCDAB is likely a functional fumarate reductase, probably in concert with the annotated frdABCD, albeit in an as yet undefined condition.

Loss of fumarate reductase activity uncouples respiration and growth: It has been almost eighty years since Loebel and colleagues formally noted the capacity of M. tuberculosis for curtailing its oxygen consumption under anaerobic or starvation conditions (Loebel et al., 1933), but a mechanism for this phenomenon is absent from the literature. Two distinct phases of adaptation to decreasing oxygen tension have been described; NPR (non-replicating persistence) stage 1—marked by the cessation of cell division at ˜1% O₂, and NRP stage 2—a quiescent state occurring below 0.06% O₂ in which biomass production ceases (Wayne and Hayes, 1996). As preservation of an electrochemical gradient is an important component of hypoxic survival, the contribution of the two sdh mutants to aerobic respiration in bioreactors operating in batch mode was assessed.

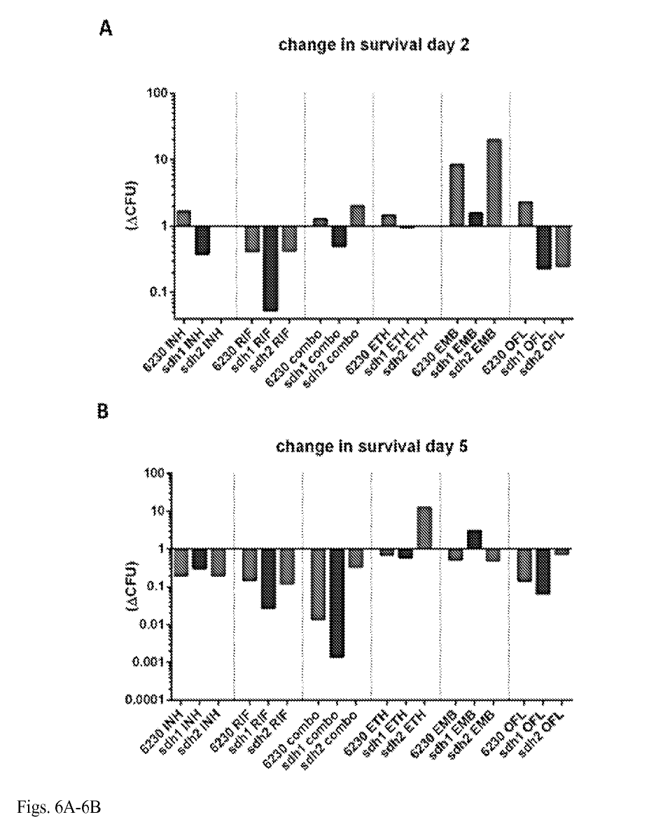

Cells were inoculated into a bioreactor system in which O₂ tension, optical density, redox midpoint potential and pH could be measured simultaneously and were followed throughout the growth curve as oxygen was depleted by the organism. The parental strain initiated down-modulation of its respiratory rate at ˜22% dissolved oxygen tension (DO), while the Δsdh1 strain continued to respire unabated until the DO was entirely depleted (FIG. 5A). This respiratory phenotype can be complemented in a strain expressing sdh1 with an integrated inducible promoter; at low levels of anhydro-tetracycline inducer (25 ng/ml—see supplemental methods). Conversely, cells harboring deletion of sdh2 consumed oxygen at a reduced rate and were able to modulate respiration as DO was depleted to ˜6%. For this strain, respiratory rate could be restored in the complemented strain using 100 ng/ml inducer.

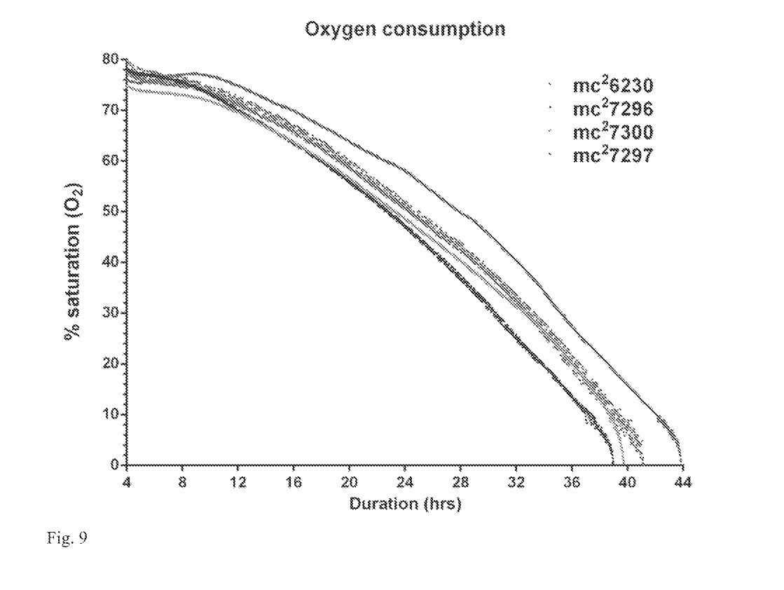

The observed respiration rate increase was not accompanied by a perceptible increase in growth rate in complete media or using glucose or glycerol as a sole carbon source (FIGS. 5B,C). Sdh1 mutants were unable to reach a similar cell density as the parent or Δsdh2 strains using succinate as a sole carbon source (FIG. 5D), as predicted from the results of the metabolome data; but Δsdh2 mutants actually reached higher densities using a fatty acid as a sole carbon source (FIG. 5E). In addition, data collected during the bioreactor experiments revealed that the initial growth rate for the sdh1 mutant is actually slightly slower in aerobic conditions (Table 1); the growth rate decreases considerably once oxygen is depleted, yet the parental strain maintains a faster growth rate than either mutant (Table 1, FIG. 9). Because the membrane potential (ΔΨ) is a component of the proton motive force (PMF), which has previously been associated with stimulation of susceptibility in persister populations (Allison et al., 2011), this study asked if differences in potential could be detected in living organisms. Membrane potential and permeability were assessed by measuring uptake of the lipophilic cation, tetraphenylphosphonium (TPP+). In both aerobiosis and anaerobic conditions, the ΔΨ was higher for the Δsdh1 mutant than wild type (Table 2), this was also the case for the Δsdh2 mutant in aerobiosis, but in hypoxia ΔΨ of Δsdh2 was considerably lower, confirming the predicted role of fumarate reduction in maintaining a membrane potential in hypoxia. The opposing effects on oxygen consumption by these enzymes implies that M. tuberculosis employs an orchestrated respiratory slowdown as oxygen levels fall; this program is initiated while oxygen is still plentiful, yet optimal growth rate is not improved by the respiratory uncoupling.

TABLE 1
Doubling times (hours⁻¹) of mc²6230 strains at 100%-1% DO (aerobic
rate), and at 1%-0% (anaerobic rate) during growth in batch culture.

| strain | mc²6230 | Δsdh1 | Δsdh2 | Δndh | ΔndhA |
|---|---|---|---|---|---|
| aerobic rate | 27.90 | 32.86 | 30.90 | 28.57 | 32.86 |
| anaerobic rate | 123.43 | 146.46 | 153.40 | 163.81 | n/a |

Respiratory control of M. tuberculosis is governed by the redox state of the menaquinone pool: it was hypothesized that the unrestrained respiration exhibited by the Δsdh1 strain was due to disruption of the redox balance of the quinone pool and sought corroborating evidence using M. tuberculosis strains harboring deletions of the type II NADH dehydrogenases ndh and ndhA. A down-modulation of oxygen consumption by these strains occurred at ˜50% and ˜10%, respectively (FIG. 4A). Complementation of ΔndhA was similar to that of the sdh enzymes with overcomplementation of O₂ consumption when ndhA was expressed episomally using a constitutive promoter, further illustrating the necessity of “fine tuning” respiratory enzyme levels. This finding suggests that enzyme activities which result in oxidation of the quinone pool serve to increase respiratory rate in the wt strain (since their deletion reduces oxygen consumption), and fumarate reduction functions as a respiratory brake during aerobiosis by an opposing reduction of the pool. The unexpected difference in DO-sensitive modulation of respiration by the type II NADH Dehydrogenases suggests a wider strategy to indirectly sense oxygen concentrations in the immediate environment and spend reducing equivalents accordingly before taking the drastic step of uncoupling biomass production from respiration.

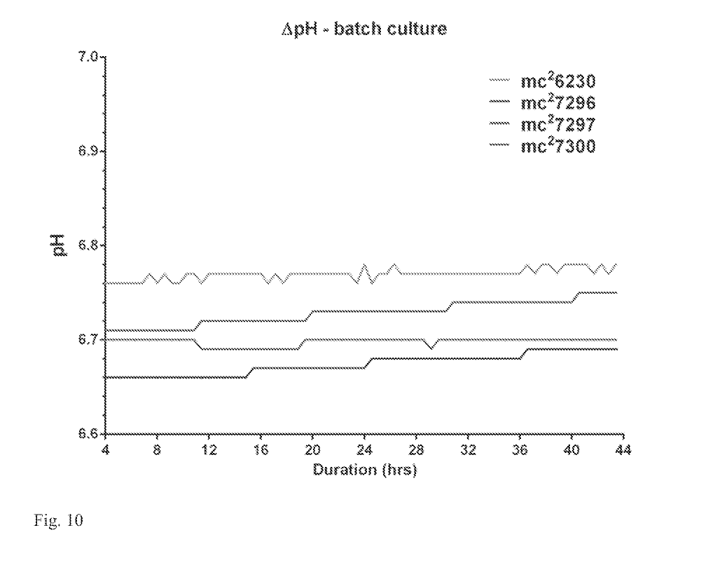

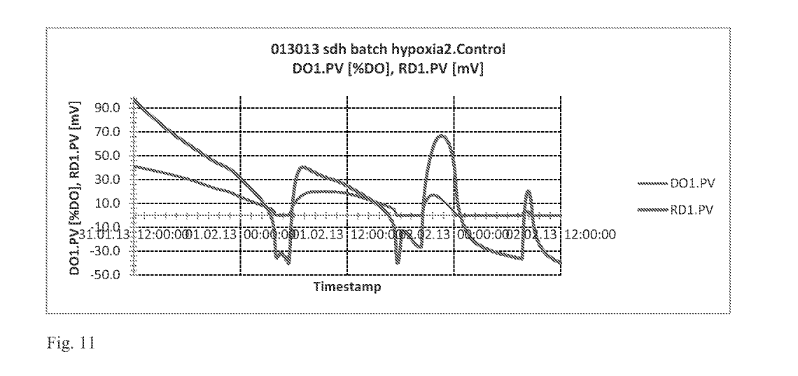

The apparent diminution of succinate oxidation in the Δsdh1 mutant during aerobiosis, and its uncontrolled respiratory phenotype alluded to an imbalance in the redox state of the menaquinone pool. To determine if this was the case, apolar extractions were performed to isolate menaquinones from cells growing at ˜1% dissolved oxygen (DO) concentration in the bioreactors (see methods). Ratios of menaquinol:menaquinone content of the parental strain was skewed toward the oxidized state in this condition, conversely the Δsdh1 mutant had higher concentrations of menaquinol, which was roughly balanced with the oxidized species. Low levels of menaquinol were also detected in the Δsdh2 mutant (FIG. 4B). In aerobically growing cells, the MK pool was found to be oxidized (ratio MK-9red/MK-9oxid=0.87), indicating an equilibrium between respiratory rate and carbon flux. Because the balance of quinone reduction can shift rapidly, further confirmation was sought by monitoring data from a probe for midpoint redox potential. Cultures were inoculated in a batch chemostat as described above but flowed compressed air into the bioreactor at 1 L/min. Using this measure, Mtb can be seen to utilize available oxygen then switch off respiration until oxygen builds up to a threshold concentration before switching on aerobic respiration again. Importantly, increases in the redox potential precede the onset of oxygen consumption by several minutes during which does not change (FIG. 10), supporting the hypothesis that oxygen consumption is managed by redox balance and not the other way around. The strain deleted for sdh2 behaves in a similar manner, but the Δsdh1 strain appears to maintain a negative midpoint redox potential and respires all available dissolved oxygen without allowing it to build up in the vessel (not shown). The above behavior is consistent with previous reports that respiratory rate can be directly controlled with first-order kinetics by the degree of reduction of the quinone pool in membrane vesicles and mitochondria (Kröger and Klingenberg, 1973; Dry et al., 1989).

Although regulation of prokaryotic central metabolic enzymes can be accomplished transcriptionally, databases containing global expression data suggest little transcriptional regulation succinate dehydrogenase enzymes. One study reported opposing expression profiles of the two annotated mycobacterial enzymes in carbon versus oxygen limiting conditions (Berney and Cook, 2010), although the fold-change in expression was minimal. Microarray analysis of transcripts was performed in both log phase aerobiosis and in twelve days of hypoxia and found sdh1 genes to be the most upregulated in the Δsdh2 mutant in hypoxia (Table 3), likely indicating some attempt at compensation. No significant regulation of sdh2 was found in the Δsdh1 strain in hypoxia. The meager differences in expression that were observed are consistent with reported data and must be considered in the light of work suggesting that primary regulation of TCA cycle activity is likely to be at the level of protein acetylation (Wang et al., 2010). The results here suggest that respiratory rate in M. tuberculosis is controlled biochemically or post-translationally, and not transcriptionally to any large extent. Management of respiration is of great importance for this organism's proclivity for survival amid a range of pathological niches.

TABLE 3
Upregulated genes - aerobic wt vs Δsdh1
Genes upregulated >2-fold

| | | |
|---|---|---|
| Condition | Gene symbol/name | Protein function |

aerobic wt vs Δsdh1

| | |
|---|---|
| Rv0318c | |
| Rv1387/PPE | PPE-family protein |
| Rv0867c | probable exported protein |
| Rv1778c | hypothetical protein |
| Rv1813c | conserved hypothetical protein |
| Rv1388/mIHF | integration host factor |

aerobic wt vs Δsdh2 - n/a
12d hypoxia wt vs Δsdh1

| | |
|---|---|
| Rv2612c/pgsA | CDP-diacylglycerol-glycerol-3-phosphate |
| Rv2886c | resolvase |
| Rv0534c/menA | 4-dihydroxy-2-naphthoate octaprenyltransferase |
| Rv0837c | hypothetical protein |
| Rv1744c | hypothetical protein |
| Rv1755c/plcD | partial CDS for phospholipase C |

12d hypoxia wt vs Δsdh2

| | |
|---|---|
| Rv0249c | probable membrane anchor protein |
| Rv0248c | probable flavoprotein subunit of Rv0247c |
| Rv3492c | conserved hypothetical protein |
| Rv0247c | probable iron-sulphur protein |
| Rv3579c | putative methyltransferase |
| Rv2052c | hypothetical protein |
| Rv3578/arsB2 | probable arsenical pump |
| Rv2886c | resolvase |
| Rv2159c | hypothetical protein |
| Rv0804 | conserved hypothetical protein |
| Rv0341 | conserved hypothetical protein |
| Rv0290 | unknown hydrophobic protein |
| Rv0394c | hypothetical protein |
| Rv1576c | hypothetical protein |
| Rv2631 | conserved hypothetical protein |

Genes downregulated >2-fold
aerobic wt vs Δsdh1

| | |
|---|---|
| Rv0219 | hypothetical protein |
| Rv3768 | hypothetical protein |
| Rv3239c | possible antibiotic efflux proteins |
| Rv1998c | conserved hypothetical protein |
| Rv2342 | hypothetical protein |
| Rv2989 | transcriptional regulator (IclR family) |
| Rv3615c | conserved hypothetical protein |
| Rv1285 | sulfate adenylate transferase, subunit 2 (cysD) |
| Rv2777c | hypothetical protein |
| Rv2987c/leuD | 3-isopropylmalate dehydratase small subunit |
| Rv2820c | hypothetical protein |

aerobic wt vs Δsdh2

| | |
|---|---|
| Rv2624c | conserved hypothetical protein |
| Rv2488c | transcriptional regulator (LuxR/UhpA family) |
| Rv0424c | hypothetical protein |

12d hypoxia wt vs Δsdh1

| | |
|---|---|
| Rv2641 | conserved hypothetical protein |
| Rv3679 | possible anion transporter |
| Rv0249c | probable membrane anchor protein |

12d hypoxia wt vs Δsdh2

| | |
|---|---|
| Rv2035 | hypothetical protein |
| Rv2204c | conserved hypothetical protein |
| Rv2913c | probable D-amino acid aminohydrolase |
| Rv3862c | hypothetical protein |
| Rv1471/trxB | thioredoxin reductase |
| Rv1875 | conserved hypothetical protein |
| Rv0331 | putative dehydrogenase |
| Rv0792c | mercuric reductase/transcriptional regulator, fusion |
| Rv3223c/sigH | ECF subfamily sigma subunit |
| Rv0767c | hypothetical protein |
| Rv2034 | transcriptional regulator (ArsR family) |
| Rv1169c | PE-family protein |
| Rv3252c | possible alkane-1 monooxygenase |
| Rv2641 | conserved hypothetical protein |
| Rv3229c/desA3 | acyl-[ACP] desaturase |
| Rv0991c | hypothetical protein |
| Rv0769 | similar to 7-alpha-hydroxysteroid dehydrogenase |
| Rv2699c | conserved hypothetical protein |
| Rv0186/bglS | [beta]-glucosidase |
| Rv1813c | conserved hypothetical protein |
| Rv2104c | conserved hypothetical protein |
| Rv2203 | hypothetical protein |
| Rv1218c | probable ABC transmembrane transport proteinportein |
| Rv3767c | conserved hypothetical protein |
| Rv0332 | hypothetical protein |
| Rv0140 | conserved hypothetical protein |
| Rv0791c | possible monooxygenasemonoxygenase |
| Rv1168c | PPE-family protein |
| Rv2642 | transcriptional regulator (ArsR family) |
| Rv1894c | some similarity to dioxygenases |
| Rv0847/lpqS | lipoprotein |
| Rv0654 | putative dioxygenase |
| Rv3418c/groES | 10 kD chaperone |
| Rv3160c | putative transcriptional regulator |

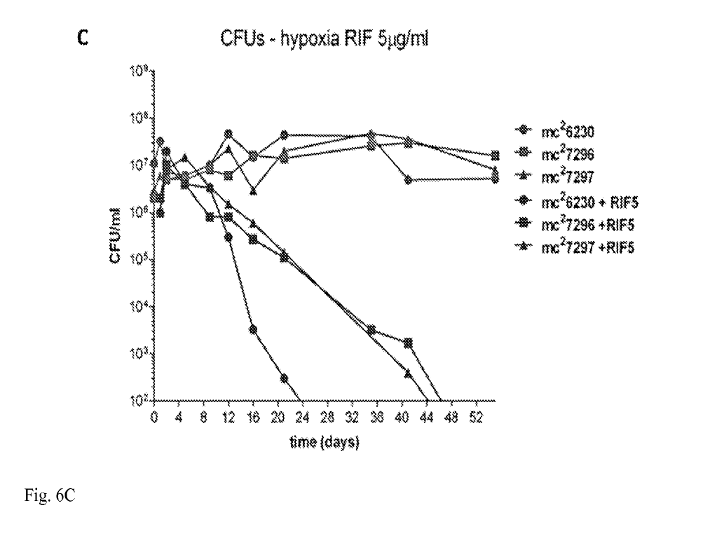

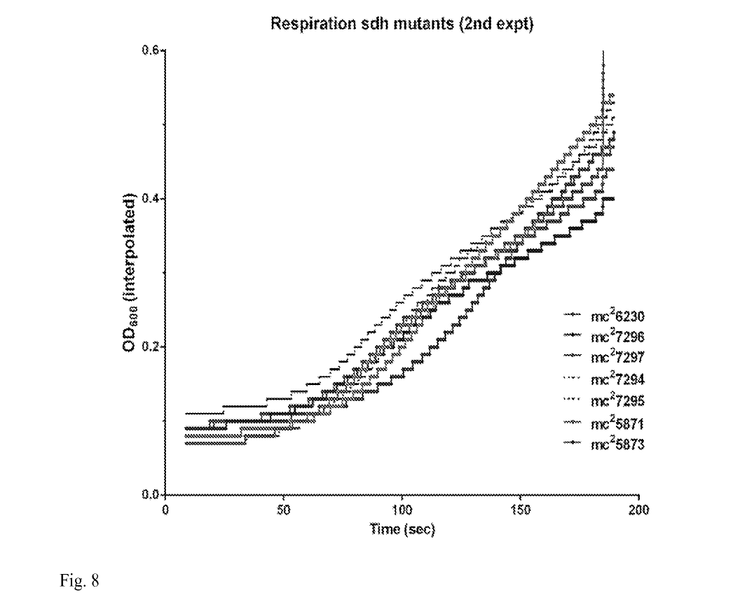

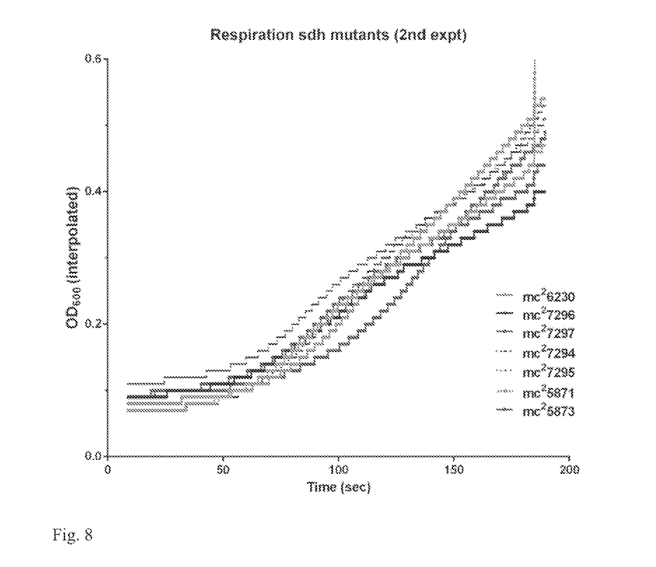

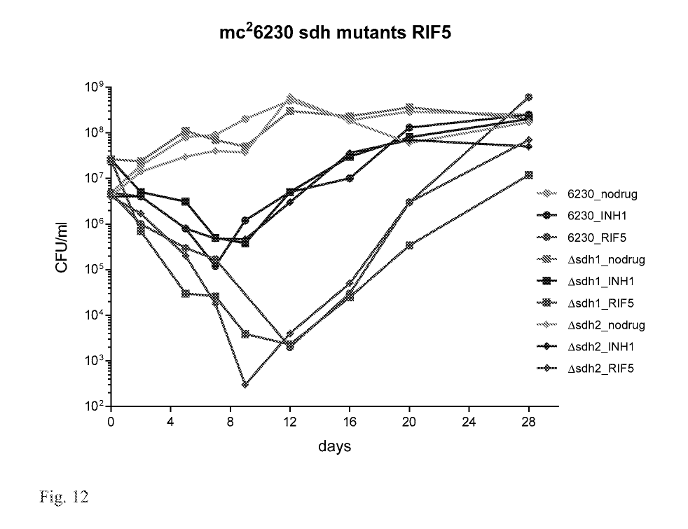

M. tuberculosis strains deleted for Rv0247c-Rv0249c show rapid kinetics of killing by front-line antituberculars: To evaluate the susceptibility profile of the M. tuberculosis Δsdh1 mutant toward other antibiotics, a number of in vitro treatments were performed in both aerobic and hypoxic conditions. Because the Δsdh1 mutant showed increased rate of killing by the combination of INH and RIF at 10×MIC, sensitivity to other tuberculosis drugs was examined by treating cells in vitro and plating CPUs over time. For several front line antibiotics, the Δsdh1 mutant was cleared more rapidly by single drugs (RIF, ETH, & OFL) at two and five days (FIGS. 6A,B), but this was not true for other single drugs (INH, STR, & EMB). Importantly, there were no profound differences in total numbers of cells killed over the duration of experiments and there were no consistent change in the time frame at which genetic mutations arose for any of the single drugs (FIG. 12). Other drug combinations also did not yield consistent differences (RIF+OFL, INH+OFL, EMB+OFL, INH+EMB, OFL+EMB; not shown). Generally, the results of kill curves were highly dependent on the history of the inoculum used for seeding the experimental samples.

Most antibiotics appear to lose efficacy on stationary phase cells or in cells in anaerobic conditions; in M. tuberculosis, rifampicin, clofazimine and pyrazinamide are exceptions. The activity of RIF on strains in hypoxic conditions was tested. In contrast to the aerobic kill curves, mutants of either Δsdh1 or Δsdh2 were killed at a reduced rate by RIF compared to the parental strain (FIG. 6C), while INH alone had no effect, and the combination of INH+RIF also showed a similar trend as RIF alone.

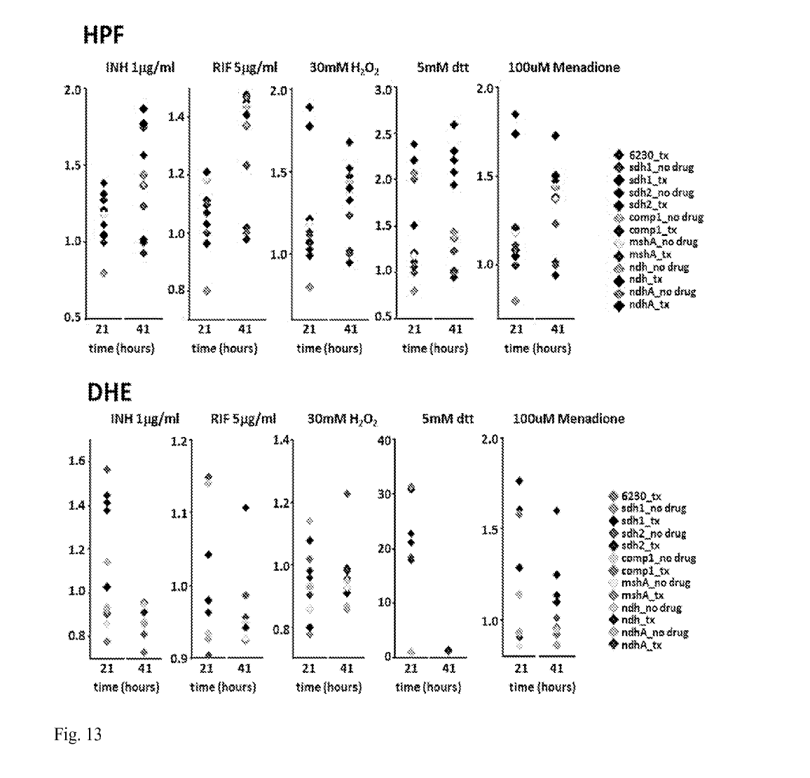

Our interpretation of this data is that respiration rates in aerobic conditions closely predict kinetics of killing for bactericidal antibiotics. In conditions where little or no oxygen is present, antibiotics targeting growth specific processes should have no effect and drugs targeting pathways important for maintenance bioenergetics should be effective. Because growth rate does not appear to be enhanced by the increased respiration of the Δsdh1 mutant (which would indicate amplified susceptibility due to faster division), another explanation was sought for the rapid killing of the Δsdh1 mutant in the potential for antibiotic-induced production of reactive oxygen species (ROS). Although higher basal levels of ROS were observed in both sdh mutants, no trend was detected in their production in response to the antibiotics tested here (FIG. 13) This does not rule out the possibility that overall ROS-induced damage is closer to a hypothetical lethal threshold in strains displaying altered respiratory kinetics.

### Example 2

Bactericidal drugs such as isoniazid or rifampicin kill 99 to 99.9% of exponentially growing Mycobacterium tuberculosis cells, yet the remaining cells initially lack resistance alleles before a drug-resistant population emerges. Herein it is disclosed that adding cysteine to isoniazid- or rifampicin-treated M. tuberculosis cultures sterilized these cultures. While cysteine failed to kill genetically drug-resistant strains, the combination of cysteine and isoniazid sterilized a rifampicin-resistant M. tuberculosis culture. This effect was not specific to cysteine, since other small thiols such as dithiothreitol or penicillamine gave the same results, but was oxygen-dependent. Increased levels of reactive oxygen species (ROS) were found in M. tuberculosis cultures treated with cysteine and isoniazid or rifampicin. This set of data along with the lack of activity under anaerobic conditions suggests that ROS play a role in the combined effect of cysteine and isoniazid or rifampicin. Administering small thiol-containing compounds with anti-tuberculosis medications can prevent the emergence of drug resistance or persisters during treatment of tuberculosis-infected patients.

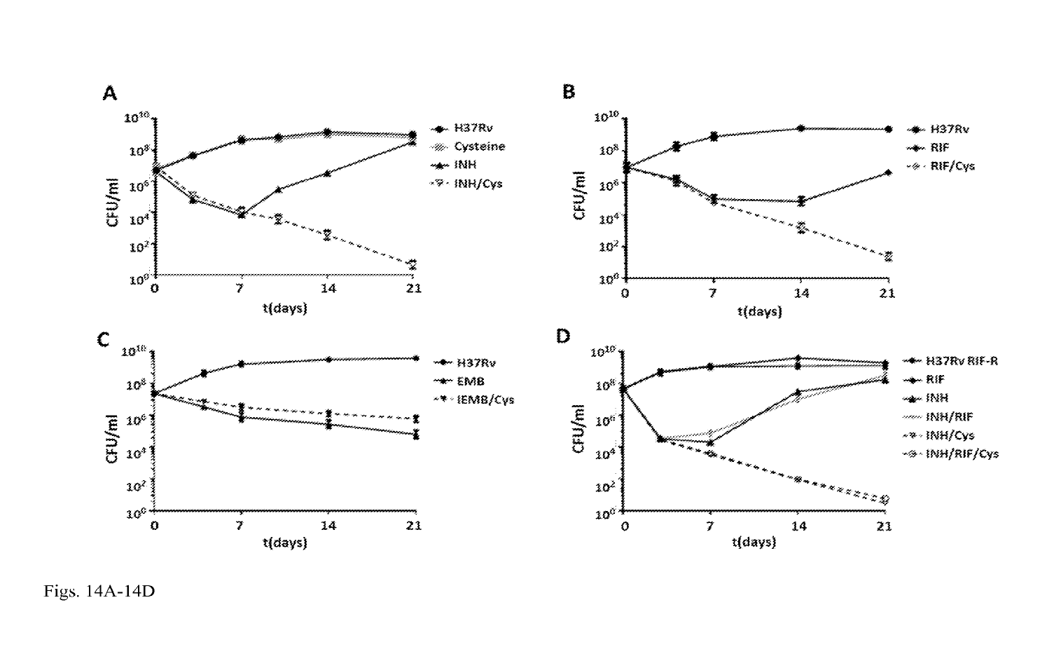

Addition of cysteine to INH-treated M. tuberculosis results in sterilization. The addition of INH (7.3 μM, 20 times the minimum inhibitory concentration (MIC)) to M. tuberculosis H37Rx results in bactericidal killing (average 3-4 log decrease in CFUs) for the first 7 days of treatment (FIG. 14A), followed by the emergence of an INH-resistant population after 10 days consisting of various katG mutants (Table 4). Low-level INH resistance was observed in M. tuberculosis mutants that failed to produce mycothiol, a cysteine reservoir, suggesting these mutants might accumulate cysteine. In order to test whether an excess in intracellular cysteine concentration might explain this resistance mechanism, increasing concentrations of cysteine (from 8 μM to 4 mM) were added to INH-treated M. tuberculosis H37Rv cultures. Interestingly, the INH-resistant population did not emerge faster with the addition of cysteine; instead the growth of INH-resistant mutants was delayed with increasing concentration of cysteine and prevented at the highest concentration of cysteine tested (4 mM). Both INH and the combination of INH and cysteine (INH/Cyst) treatments of M. tuberculosis followed the same kinetics of killing for the first 7 days, but then diverged when the INH-resistant population starts emerging in the INH-treated M. tuberculosis culture (FIG. 14A). Bacterial counts after 3 weeks of INH/Cyst treatment of M. tuberculosis ranged between 0 and 10 colony-forming units (CFU)/ml, representing a 6 log killing of the initial culture (FIG. 14A). This increased killing was not due to a bactericidal effect of cysteine itself, since growth of M. tuberculosis was not affected by the addition of cysteine alone. This was not specific to M. tuberculosis H37Rv either. The same sterilizing activity was observed when M. tuberculosis Beijing or M. tuberculosis CDC1551 was treated with a combination of INH (7.3 μM) and cysteine (4 mM) (data not shown).

The effect of cysteine is not restricted to INH. To test whether the effect of INH/Cyst was specific to INH, M. tuberculosis cultures were co-treated with cysteine and two other first-line TB drugs: RIF and ethambutol (EMB). Treatment of M. tuberculosis with RIF (1.2 μM, 10 times the MIC) resulted in a 3 to 4 log decrease in CFU after 2 weeks followed by an increase in CFU due to the emergence of RIF-resistant mutants (FIG. 14B). When cysteine was added to M. tuberculosis in combination with RIF, a 6-log decrease in CFUs was observed (FIG. 14B). However, a different pattern was observed for EMB (FIG. 14C). Treatment of M. tuberculosis with EMB (0.5 mM, 20 times the MIC) and with or without cysteine resulted in a very slow decrease in CFU logs after 3 weeks). No emergence of EMB-resistant mutants was observed. The colonies obtained after 3 weeks of EMB treatment were all EMB-susceptible, while the majority of colonies isolated after 3 weeks of INH or RIF treatment were INH-resistant or RIF-resistant, respectively. This suggested a correlation between the emergence of a drug-resistant population and the beneficial effect of adding cysteine to a drug treatment. Since it was observed that cysteine did not increase the killing of M. tuberculosis by ETH, the colonies isolated from ETH-treated M. tuberculosis cultures at different time points were also checked for ETH resistance and were found to be ETH-susceptible as well.

A free thiol group is required for the sterilizing activity in combination with INH. Cysteine is a small amino acid with a mercaptomethyl group attached to the amino acid backbone. Therefore, it was asked which part of the chemical structure of cysteine was required for this increased bactericidal killing of M. tuberculosis with INH or RIF. First, since cysteine possesses a chiral center, it was examined if D-cysteine was as potent as L-cysteine in killing M. tuberculosis in combination with INH or RIF; and found that they had the same effect. Then, chemically similar amino acids were tested such as serine, in which the thiol group of cysteine is replaced by a hydroxyl group, and methionine, in which the thiol group is methylated. These 2 amino acids, at the same concentration as cysteine (4 mM), did not increase the killing of M. tuberculosis when combined with INH (Table 5). The growth kinetics of M. tuberculosis treated with a combination of serine or methionine and INH were similar to that of INH treatment alone, with the emergence of a growing population after 14 days. This suggested that it was the free thiol in cysteine that was crucial for the increased bactericidal activity. This hypothesis was further validated by the testing of a variety of small thiol compounds (Table 5). The emergence of an INH-resistant population was prevented only when M. tuberculosis was treated with a combination of INH and a compound having a free thiol group. There was one noticeable exception though: N-acetylcysteine (NAC) did not prevent the emergence of a drug-resistant population despite its free thiol group. The amino acid backbone was not required either since the addition of dithiothreitol (DTT) to or RIF-treated M. tuberculosis cultures also resulted in sterilization of the cultures after 3 weeks. Interestingly, DTT, at just half the concentration used for cysteine (2 mM), sterilized M. tuberculosis cultures when combined with INH. This confirmed the crucial role of the free thiol group, since DTT possesses two free thiol groups whereas cysteine has only one. The remainder of this report will focus on cysteine as a physiologically relevant thiol delivery agent.

TABLE 4
katG mutations identified in 2-5 colonies isolated from
M. tuberculosis cultures treated with INH for 21 days.

| INH-treated M. tuberculosis | katG mutation |
|---|---|
| Culture A | g502c (A168P) |
| | g1663c (A555P) |
| | Duplication of region 2457-2473* |
| Culture B | g332a (G111D) |
| | c908a* (Stop codon) |
| | g1514a (Stop codon) |
| Culture C | deletion c1200 (frameshift/stop codon) |
| | t1880c (L627P) |
| | t2182c (W728R) |
| Culture D | g632c (S211T) |
| | g952t (Stop codon) |
| Culture E | c695t* (P232L) |
| | t982g (W328G) |

*mutation found in more than one clone.

TABLE 5
Compounds tested for activity when combined with INH.

| | | Activity |
|---|---|---|
| Compound | Structure | with INH |
| L-Cysteine | 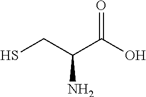 | + |
| D-Cysteine | 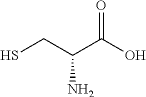 | + |
| L-Serine | 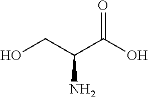 | − |
| L-Methionine | 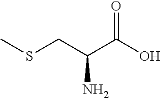 | − |
| L-Cysteine methyl ester | 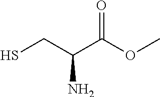 | + |
| (R)-2-oxathia- zolidine-4- carboxylic acid | 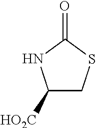 | − |
| L-Penicillamine | 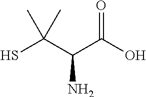 | + |
| S-Acetamido- methyl-L- cysteine | 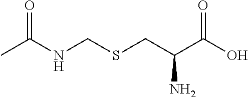 | − |
| Homocysteine | 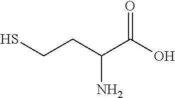 | + |
| N-Acetyl- cysteine | 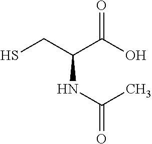 | − |
| S-(2-Amino- ethyl)-L- cysteine | 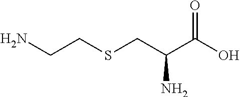 | − |
| Dithio- threitol | 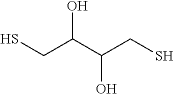 | + |

Combining cysteine with specific TB drugs kills drug-resistant M. tuberculosis. One of the challenges facing TB eradication is the rapid spread of drug-resistant TB strains. Since one of the possible mechanisms by which cysteine or small thiols work might be by killing INH-resistant mutants that emerge during INH treatment of M. tuberculosis, it was tested whether the activity of cysteine against three INH-resistant M. tuberculosis strains [H37Rv inhA S94A (Vilchèze et al., 2006), H37Rv PmabAinhA c-15t (Vilchèze et al., 2006), and H37Rv katG g371del]. The strains were grown with cysteine (4 mM), INH (7.3 μM), or INH/Cyst. Growth was not impaired for the INH-resistant strains in those three conditions.

Although the combination of cysteine and INH did not inhibit growth of INH mono-resistant M. tuberculosis strains, it was investigated whether a combination of cysteine and a TB drug might still be beneficial in treating drug-resistant M. tuberculosis strains. The INH-resistant H37Rv katG g371del was treated with RIF (1.2 μM), cysteine (4 mM), and RIF/Cyst, and a RIF-resistant M. tuberculosis strain (H37Rv rpoB H445R, RIF MIC>15 μM), with INH (7.3 μM), cysteine (4 mM), and INH/Cyst. In both cases, the combination of cysteine with RIF or INH prevented the growth of the respective drug-resistant mutant populations. To further assess the effect of this combination, CPUs were determined for the RIF-resistant H37Rv rpoB H445R strain treated with INH (7.3 μM), RIF (1.2 μM), cysteine (4 mM), and INH/Cyst combined or not with RIF (FIG. 14D). H37Rv rpoB H445R grew well in the presence of RIF or cysteine alone (data not shown). The growth pattern of H37Rv rpoB H445R, treated with INH was similar to the INH-treatment of a wild-type M. tuberculosis strain. INH/Cyst with or without RIF resulted in a 6-log killing after 3 weeks, suggesting that the addition of cysteine would be beneficial in the treatment of M. tuberculosis strains resistant to INH or RIF.

Cysteine reduces the frequency of INH mutation. Cysteine has previously been described as the most anti-mutagenic of all the amino acids (Roy et al., 2002). Cysteine, with its free sulfhydryl group, can scavenge radicals or electrophilic molecules and therefore act as an anti-oxidant or anti-mutagen. To assess if cysteine caused a reduction in the generation of spontaneous resistant mutants in INH- or RIF-treated M. tuberculosis, the frequency of mutations to INH or RIF was measured in independent M. tuberculosis M37Rv cultures grown with and without cysteine (Table 6). Growing M, tuberculosis with cysteine caused up to a 10-fold decrease in the frequency of spontaneous INH-resistant mutants, but only a 3-fold decrease in the frequency of spontaneous RIF-resistant mutants. Transcriptional profiling was used on 4-day-old cultures of M. tuberculosis H37Rv treated with INH with and without the addition of 4 mM cysteine to explore the anti-mutagenic effect of cysteine combined with INH. Very few genes were significantly up-regulated and none were down-regulated 1.4 fold or more (Table 4). Genes that were statistically significantly up-regulated 1.4 fold or more in INH-cysteine-treated cultures versus those treated with INH alone were genes that had been reported to be up-regulated in the presence of high levels of copper (Ward et al., 2008). The most up-regulated gene, 1pqS, is located upstream of cysK2, which encodes the cysteine synthase A. Previous work has shown that cysK2 was up-regulated 20-fold in M. tuberculosis grown in media containing high concentration (0.5 mM) of CuCl₂ (Ward et al., 2008). The same study (Ward et al., 2008) also showed that Rv0967, a gene encoding a copper-sensitive repressor, and the hypothetical protein Rv0190 were up-regulated 30- and 8.5-fold, respectively, in media containing 0.5 mM of cupric chloride. Cupric ion can be reduced by cysteine to form cuprous ion, which can react with hydrogen peroxide to generate hydroxyl radicals that can damage DNA. Thus, it was hypothesized that high concentrations of cysteine trigger a divalent cation-induced stress which could result in the formation of ROS.

TABLE 6
Frequency of mutations of M. tuberculosis
H37Rv grown with or without cysteine.

Frequency of
mutation for

| Culture # | Cysteine (4 mM) | INH | RIF |
|---|---|---|---|
| 1 | − | 4 × 10⁻⁶ | nd |
| 2 | − | 1.4 × 10⁻⁵ | nd |
| 3 | − | 2 × 10⁻⁶ | 7 × 10⁻⁸ |
| 4 | − | 1.6 × 10⁻⁵ | 5 × 10⁻⁸ |
| Average | − | 9 × 10⁻⁶ | 6 × 10⁻⁸ |
| 5 | + | 1 × 10⁻⁷ | nd |
| 6 | + | 1 × 10⁻⁸ | nd |
| 7 | + | 2 × 10⁻⁶ | 2 × 10⁻⁸ |
| 8 | + | 7 × 10⁻⁷ | 2 × 10⁻⁸ |
| Average | + | 7 × 10⁻⁷ | 2 × 10⁻⁸ |

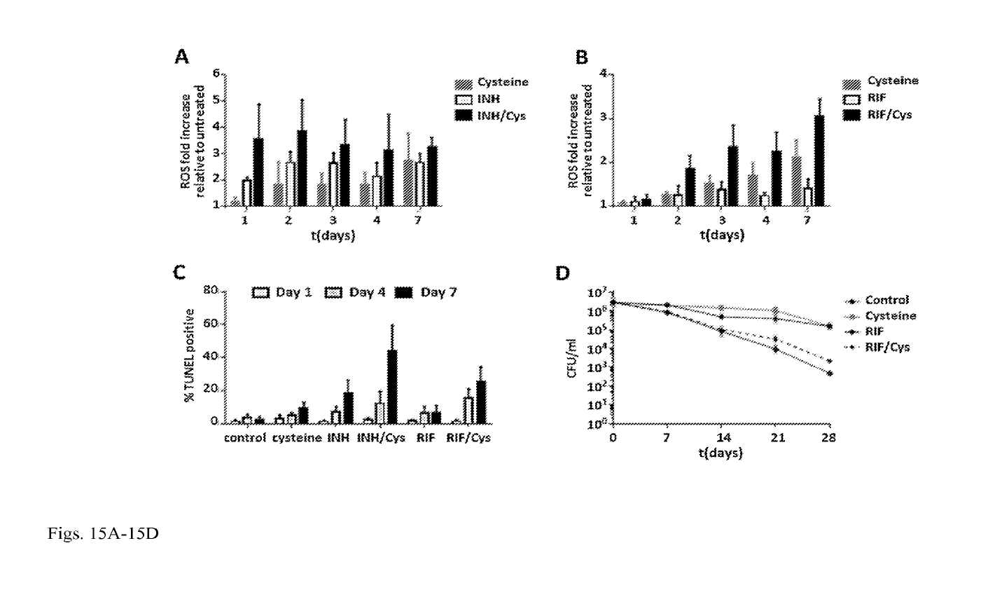

The combination INH-cysteine increases oxidative damage. Although our levels of up-regulation were much lower than those observed by Ward et al (Ward et al., 2008), it was examined whether INH/Cyst- or RIF/Cyst-treated M. tuberculosis cultures could generate reactive oxygen species (ROS) that would result in double-stranded (ds) DNA breaks and DNA damage. For safety purposes, these experiments were performed with M. tuberculosis mc26230, a non-pathogenic H37Rv ΔRD1 ΔpanCD laboratory strain that can safely be used in a BSL2 laboratory. ROS levels and DNA breaks were measured in M. tuberculosis treated with cysteine, INH, RIF, INH/Cyst and RIF/Cyst during a period of seven days post-treatment by flow cytometry. Although the INH and cysteine treatment showed a clear increase in ROS formation in the first 24 hr compared to cysteine or INH alone, these differences disappeared at longer time points (FIG. 15A). A different pattern was observed for the combination of RIF and cysteine. While ROS production was at its highest levels after 24-48 h in the INH/Cyst samples (four-fold compared to untreated), there was no change in ROS level after 24 h compared to untreated sample in the RIF or RIF/Cyst samples (FIG. 2B). A three-fold increase in ROS concentration compared to untreated was observed after seven days of RIF/Cyst treatment. This increase in ROS production correlated with a dramatic increase in the level of ds DNA breaks, especially in the INH/cysteine treated samples with up to 60% of cells showing signs of ds DNA breaks after seven days (FIG. 15C).

To correlate the enhanced ROS production upon combination of cysteine with INH or RIF to bactericidal activity, M. tuberculosis mc26230 was treated with cysteine, RIF and RIF/Cyst in an anaerobic chamber. For this experiment, RIF was chosen as it retains bactericidal activity under anaerobic conditions while INH does not (Piccaro et al., 2013). Under total absence of oxygen, the addition of cysteine to RIF-treated M. tuberculosis did not increase killing of the bacterial population (FIG. 15D).

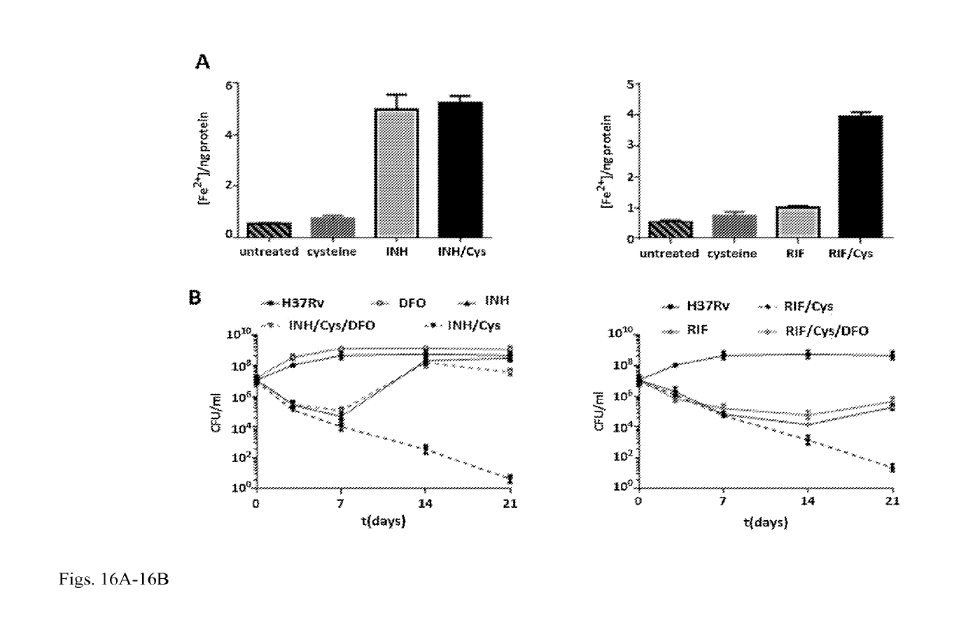

The transcriptional data suggested that copper might be involved in the INH/Cyst activity. To test whether the INH/Cyst bactericidal activity correlated with copper ion level, the effect of the copper (1) chelator neocuproine was tested in the presence of cysteine and INH. Although the lowest concentration of neocuproine tested (4 μM) had no impact on the growth of M. tuberculosis H37Rv, neocuproine unexpectedly showed activity in combination with INH (7.3 μM) alone resulting in a 5-log decrease in CFUs after seven days of treatment. A similar pattern was observed for RIF (1.2 μM) and neocuproine with a 5-log decrease in CFUs after 14 days. Since neocuproine was bactericidal when combined to INH or RIF, we could not test whether neocuproine would alleviate the effect of cysteine in INH- or RIF-treated M. tuberculosis cultures. The data indicate that INH/Cyst and RIF/Cyst treatments lead to ROS production and DNA damage. This might occur via the Harber-Weiss/Fenton reactions, which generate ROS by reducing cupric or ferric ion to produce cuprous or ferrous ion (Wardman & Candeias, 1996). Ferrous ion (or cuprous ion) can then react with oxygen to form superoxide, which by dismutation will produce hydrogen peroxide. Hydrogen peroxide will next reduce ferrous ion (or cuprous ion) and form hydroxyl radical and ferric (or cupric) ion. Since the transcriptional data showed only a very small up-regulation of genes responding to copper level and cysteine is known to reduce ferric ion to ferrous ion very rapidly in Escherichia coli leading to oxidative DNA damage (Park & Imlay, 2003), it was tested whether iron also plays a role in the INH/Cyst and RIF/Cyst activities. The intracellular levels of ferrous ions were measured in M. tuberculosis treated with cysteine, INH, RIF, INH/Cyst or RIF/Cyst for 3 days and found to be up to ten times higher in the INH/Cyst- and RIF/Cyst-treated samples than in the untreated sample (FIG. 16A). Surprisingly, the ferrous ion concentration was also very high in the INH-treated sample. This might correlate with the ROS production which at 3-day post-treatment was similar between the INH- and INH/Cyst-treated samples (FIG. 15A). To test whether this increase in ferrous ion concentration correlated with the bactericidal activity of the INH/Cyst and RIF/Cyst combination, the effect of the iron chelator deferoxamine (DFO) on M. tuberculosis treated with cysteine and INH or RIF was then examined. It is also shown herein that DFO could reverse the killing of M. tuberculosis by a vitamin C-induced Fenton reaction (see also Vilchèze et al., 2013). The addition of DFO to INH/Cyst- or RIF/Cyst-treated M. tuberculosis cultures resulted in killing curves similar to the ones obtained for INH- or RIF-treated M. tuberculosis (FIG. 16B). The effect of cysteine was abolished when DFO was added to the INH/Cyst or RIF/Cyst combination.

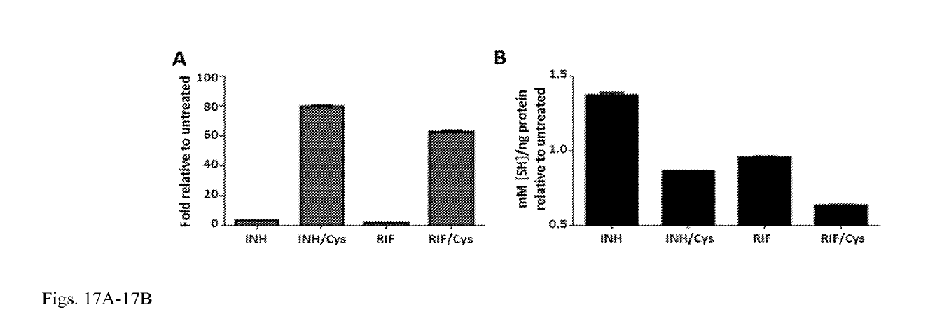

The first step in the Harber-Weiss/Fenton reactions is the reduction of ferric ion or cupric ion to produce ferrous ion or cuprous ion and requires a reductant to initiate the reaction. Here, the reductant is cysteine and during the reduction of ferric or cupric ion by cysteine, cysteine will be oxidized to cystine. To test whether cysteine was indeed oxidized to cystine, cell pellets extracted from M. tuberculosis treated with INH (7.3 μM), RIF (1.2 μM), cysteine (4 mM), or the combination were analyzed by LC-MS to determine the levels of cystine in the samples. The cystine concentration was 100-times higher in the cysteine-treated samples than in the untreated sample (FIG. 17A) and no increase in cysteine levels were observed despite the addition of 4 mM of cysteine to the samples. To confirm that the levels of cysteine were not higher in the cysteine-treated samples, the levels of free thiols were next measured in the cell pellets of M. tuberculosis treated with INH (7.3 μM), RIF (1.2 μM), cysteine (4 mM), or the combination for 24 h (FIG. 17B) using Ellman's Reagent. The levels of free thiols were in fact lower in the cysteine-treated M. tuberculosis samples confirming that the addition of 4 mM of cysteine did not increase the thiol level in the INH- or RIF-treated M. tuberculosis cultures.

### Example 3

Vitamin C (VC) produces ROS in M. tuberculosis. The ability of VC to reduce ferric ions to ferrous ions is well known and is the basis of its pro-oxidant property. The production of ferrous ions leads to the generation of ROS (superoxide, hydrogen peroxide and hydroxyl radicals) via the Harber-Weiss and Fenton reactions, which can result in DNA damage. To test if VC activity against M. tuberculosis could be a consequence of this mechanism, ROS accumulation and DNA damage were measured in M. tuberculosis cultures treated with VC using flow cytometry. Up to threefold increase in total ROS was observed in M. tuberculosis treated with 4 mM VC, This increase was dependent on the concentration of VC; 0.4 mM VC did not substantially alter total ROS concentration relative to the untreated sample. As a control, the fat-soluble antioxidant vitamin E (VE) was also tested. In contrast to VC, VE had lowered ROS level than the untreated samples after 3 days. To assess the DNA damage potentially caused by ROS accumulation, DNA fragmentation was measured in M. tuberculosis treated with VC or VE using the terminal deoxynucleotidyltransferase-mediated dUTP-biotin nick end labelling (TUNEL) assay. The percentage of cells with DNA breaks in M. tuberculosis treated with 4 mM VC increased over time to reach 17-25% after 9 days, tenfold higher than in the untreated culture or in the culture treated with VE.

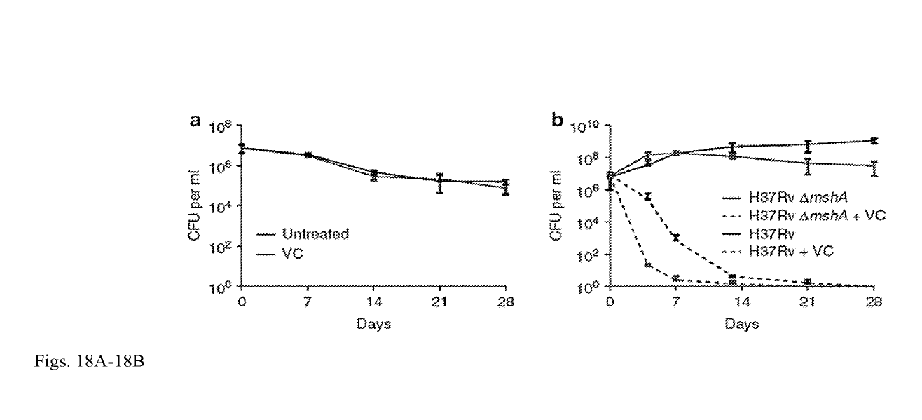

To further test if this increase in ROS production was one of the mechanisms for the bactericidal activity of VC in M. tuberculosis, cultures were treated with VC in an anaerobic chamber in which the level of oxygen is kept below 0.001%. VC had no effect on M. tuberculosis viability in this condition (FIG. 18A). Accordingly, vitamin C can be used as a respiratory stimulator in combination with an anti-tuberculosis drug as a treatment for tuberculosis infections and TB.

### DISCUSSION

### Example 1

An unfortunate consequence of long duration antibiotic therapy is patient noncompliance, an unacceptable outcome for individuals harboring a communicable infectious agent, as it facilitates the emergence of drug resistance. The work presented here directly assesses the contributions of specific genetic factors which determine kinetics of killing of Mycobacterium tuberculosis by common front-line antibiotics. In lieu of a technology for physical isolation of persister cells without disrupting their theoretically labile metabolic state, negative genetic screen (TraSH) was performed for genes whose loss would confer a survival disadvantage under combination antibiotic pressure. The use of multiple antibiotics would decrease the noise contributed by genetically resistant mutants, and that the choice of INH and RIF would provide a relevant selective pressure as these are the drugs used in the continuation phase of standard antitubercular chemotherapy. Any gene which contributes to longevity during a short course in vitro chemotherapy regimen could shed light on the physiological state of M. tuberculosis persisters and guide research efforts in rapid elimination of this population from infected hosts. These two drugs were chosen for their proven efficacy in sterilization of TB as well as their non-overlapping mechanisms and pharmacodynamics profiles which indicate concentration and exposure-dependent killing respectively (Jayaram et al., 2003; Jayaram and Shandil, 2004; Vilchèze and Jacobs, 2007). Results suggested that a number of non-essential components of the electron transport chain could contribute to this resistance to primary killing, thus emphasizing the bioenergetic underpinnings of drug tolerance. The appearance of one of the annotated succinate dehydrogenase enzymes—but not the other—in the resulting data suggested a way to examine the link between central carbon metabolism and electron transport. Isogenic strains containing a deletion of this operon were found to be cleared more rapidly by combination chemotherapy.

The highly flexible nature of the aerobic and anaerobic respiratory enzymes of this organism has been reviewed elsewhere (Bavesh D. Kana et al., 2009), but genome annotation includes two predicted succinate dehydrogenase and one predicted fumarate reductase operon. Genetic manipulation of M. tuberculosis followed by an intracellular metabolomic approach allowed us to probe the functions of the two annotated Sdh enzymes in a physiologically relevant context, using extracts from living organisms. These studies supported the predicted role of Rv0249c-Rv0247c as a succinate dehydrogenase during aerobiosis, and indicated fumarate reductase capacity by the operon encoded by sdhCDAB. These enzymes were found to strongly influence aerobic respiration, and deletion of sdh1 resulted in a consistent increase in the rate of oxygen consumption, but did not result in more rapid growth. Conversely, deletion of the operon containing sdh2 led to a diminished respiratory capacity, a pattern which precisely matched death curves plotted for these strains for the combination therapy.

The oxygen consumption profiles of the two sdh mutants revealed another interesting aspect of mycobacterial physiology, a downshift of respiratory activity was engaged by the parental strain in the range of 30-40% dO₂. Current understanding of the mechanics of the decline in respiratory activity exhibited by M. tuberculosis upon adaptation to anaerobic conditions has been guided by analysis of the transcriptome of cells as they pass into hypoxia in various models (Voskuil, 2004; Shi et al., 2005; Berney and Cook, 2010). M. tuberculosis accomplishes gross control of respiration by depriving the cytochrome oxidoreductase of menaquinol and demonstrate how carbon flux through the TCA cycle integrates into this signal to couple growth and electron transport. Importantly, this modulation in the of rate of oxygen consumption occurs long before O₂ becomes limiting for growth (Cox and Cook, 2007). The data suggests that this does not reflect a strategy for diversion of carbon into storage molecules in preparation for a resource limited state , but rather a tuning of redox homeostasis to maintain an optimal growth rate (Table 1).

A simple mechanistic explanation for this phenomenon is favored and is consistent with the structural studies of the cytochrome oxidase complex (Zhang et al., 1998; Hunte et al., 2003) and the progression of the Q-cycle (Mitchell, 1975). Organisms will respire at optimal rates with a balanced quinone pool in which quinol is present in sufficient concentration to immediately occupy the center P of the cytochrome oxidase complex; but when quinol is limiting in an oxidatively skewed pool, respiration will proceed at a less-than optimal rate. (FIG. 4B) shows that whereas the wt strain has a largely oxidized quinone pool at 1% DO, the Δsdh1 mutant maintains a balanced pool, resulting in unchecked oxygen consumption. These data support a mechanism for respiratory downshift in wt MTB that works as follows: as oxygen concentration decreases, succinate oxidation also decreases leading to a buildup of succinate (hypoxic cells accumulate a sevenfold increase in intracellular concentration) This ‘unrespired’ succinate does not contribute to the reduction of membrane quinones and as the ratio of quinol:quinone increases from the activity of other electron donors, the cytochrome oxidoreductase is deprived of its substrate, thus decreasing the rate of oxygen consumption. This mechanism would be highly dependent on quinone content and would benefit from a method to rapidly analyze quinone redox status with minimal invasion and with no potential for unintended. experimentally induced oxidation. Nonetheless, published measurements of ubiquinone in E. coli show a similar trend (Bekker et al., 2007) in quinone poise as oxygen decreases.

In the related saprophyte Mycobacterium smegmatis, the cytochrome bcc complex and the aa3-type cytochrome c oxidase are physically associated by hydrogen bonds; and O₂ reduction by isolated membranes could be disrupted using Antimycin (a known Qi-site inhibitor) (Megehee et al., 2006). There was no cidal activity by Antimycin on M. tuberculosis at >180 mM, but strong bactericidal activity of sodium azide at 10 mM (not shown), confirming that the cytochrome oxidase complex is plays an essential role in aerobiosis. This supports the hypothesis of direct modulation of O₂ reduction via the redox poise of the quinone pool, which might be rate-limiting in a niche in which soluble cytochrome c (or indeed, menaquinone) could be easily oxidized by phagocytic reactive species (Smith et al., 1974; Roos et al., 1984). It remains to be seen if oxygen consumption can be controlled by skewing redox balance in organisms which are known to use soluble cytochromes c, or have physically separated reductase and oxidase complexes.

An interesting implication of the respiratory phenotype is that fumarate reduction occurs throughout the growth curve to some degree and offsets succinate oxidation to a significant degree even during aerobiosis. The necessity for members of the mycobacteria to maintain two functioning copies of the frd enzyme (sdhCDAB & frdABCD) may be an indication of a metabolic plasticity which enables them to simultaneously utilize multiple carbon sources with different oxidation states (De Carvalho et al., 2010) and divert this carbon either into biomass production or storage molecules during growth phases (Borisov et al., 2011), or into energy production for maintenance of proton motive force (PMF) and repair during non-growth states. The rerouting of a portion of carbon flux into the reductive arm of the TCA cycle (Watanabe et al., 2011) indicates the involvement of fumarate reductase activity in hypoxia; no hypoxic survival defect was reported in that work and here there are long term survival defects only after fifty days in culture alluding to the contribution of other pathways in anaerobic respiration. Compensation of Frd catalysis by the remaining enzyme remains a possible explanation, and further genetic analysis will need to be performed to establish this, but thus far it has not been possible to isolate transductants for double knockouts of sdh1 & 2.

The bioenergetic adaptation which sustains M. tuberculosis during latency has been of great interest to researchers for decades. The putative site in which latent tuberculosis organisms persist, avoiding immune surveillance and maintaining undetectable cell numbers is presently unknown. Several hypotheses have been suggested including the necrotic centers of granulomas (Gomez and McKinney, 2004; Barry et al., 2009), adipocytes (Neyrolles et al., 2006), and recently mesenchymal stem cells (Das et al., 2013). This latter work is especially interesting as new data suggests that modulation of the intracellular redox in stem cells and cancerous cells which result in an oxidative environment may serve to inhibit mitochondrial respiration, thus limiting ROS damage to progenitor mitochondrial DNA (Noble and Smith, 2003; Ogasawara and Zhang, 2009). This raises the possibility that that same oxidized environment would serve to inhibit respiration by intracellular M. tuberculosis leading to a persistent state. Since cells in NRP-2 maintain an energized membrane, and are notably tolerant to single antibiotics but retain sensitivity to some combinations (Filippini et al., 2010), it is plausible that both persistence and phenotypic drug tolerance are a function of the oxidative state of the milieu, and both phenotypes are the result of reduced respiratory flux. The presence of adequate oxygen atone is not sufficient to stimulate respiration; redox homeostasis must be restored before it can reach optimal levels and the cell can take advantage of the energetic benefit of oxygen as its terminal electron acceptor.

Finally, bacterial persisters appear to be present in all aerobic species, and screens have thus far been unable to unearth a common mechanism. It is becoming increasingly apparent that this phenotype is not the result of a single gene but the result of a program encompassing metabolic responses to conditions which alter cellular bioenergetics. A number of genes have been found in screens for strains producing proportionally altered numbers of persisters. Among those implicated are: glpD (which reduces quinones in its catalysis of glycerol 3-phosphate to glycerone phosphate), TA systems such as tisAB (a toxin which when overexpressed may lead to decreased membrane potential), tnaA (which cleaves L-tryptophan to indole, pyruvate and NH4+), phoU (a negative metabolic regulator), sucB (catalyzes the conversion of 2-oxoglutarate to succinyl-CoA and CO₂, producing NADH), and ubiF (which hydroxylates the benzene ring in biosynthesis of ubiquinol—with its homolog visC,) (Spoering et al., 2006; Li and Zhang, 2007; Unoson and Wagner, 2008; Dörr et al., 2010; Ma et al., 2010; Kint et al., 2012; Vega et al., 2012). The underlying mechanistic basis for the influence of these genes in increasing persister fractions is through their influence on respiratory rate. Additionally, it was found that metabolites feeding into glycolysis eliminate the persister fraction by stimulating PMF (Allison et al., 2011), but that this killing could occur anaerobically by the addition of nitrate to fermentable carbon sources, and abolished by addition of CCCP. In short, persistent fractions represent non-respiring cells, which are eliminated more slowly because carbon utilization is being directed more toward maintenance functions and less toward anabolic processes important in growth (which would render those target enzymes essential).

There is now widespread acknowledgement of the fact that reduction in the duration of TB chemotherapy could be achieved by finding ways to target dormant or persistent cells. Toward that goal, recent drug library screens have focused on finding compounds which target essential functions in non-replicating M. tuberculosis. An exciting example of the success of this approach has been borne out recently with FDA approval of the diarylquinolone Bedaquiline, an ATP-synthase inhibitor which is effective against dormant bacilli (Andries et al., 2005; Koul et al., 2008; Diacon and Pym, 2009), lending credence to the idea that non-replicating cells still remain susceptible to inhibitors of the respiratory chain. The rapidity of killing by some front-line antitubercuals can be improved by removing a metabolic block on TB respiration imposed by the contending action of the aerobic succinate dehydrogenase and fumarate reductase activities. Thus, progress toward the goal of shortening chemotherapy can be served by searching for enhancers of respiration, which would likely make cells more susceptible to existing treatments and reduce the numbers of organisms which are shifted to a persistent state.

### Example 2

With regard to the results showing a synergistic effect of cysteine observed in combination with the first-line TB drugs INH or RIF, an important effect observed is the generation of ROS. It was shown that ROS concentrations are higher in the INH/Cyst- or RIF/Cyst-treated M. tuberculosis leading to DNA damage. It was also also shown that vitamin C generates ROS production in M. tuberculosis leading to redox alteration, membrane disruption and ultimately cell death (also see Vilchèze et al., 2013). Hydrogen peroxide in the presence of metal ions such as Fe²⁺ or Cu⁺ can form hydroxyl radicals, which can fragment DNA. The microarray experiment suggested that the addition of cysteine to INH in M. tuberculosis cultures correlate with increased expression of copper-responsive genes. Although copper levels were not measured in the samples, an increase in intracellular iron concentration was seen in the samples treated with INH, INH/Cyst or RIF/Cyst as well as a loss of synergistic activity when the iron chelator deferoxamine was added to a culture. When the levels of ROS in M. tuberculosis were measured, up to a five-fold increase in ROS production was observed within 24 hours in the INH/Cyst samples while RIF/Cyst treatment required a longer exposure to generate higher levels of ROS. In contrast, the increase in DNA breaks was remarkable in M. tuberculosis treated with INH and cysteine after 7 days, when the kinetics of killing between M. tuberculosis treated with INH and M. tuberculosis treated with INH and cysteine start to diverge. Furthermore, the synergistic activity of cysteine was not observed in cultures grown under anaerobic conditions. All these observations suggest that combining INH and cysteine in M. tuberculosis results in an oxidative burst leading to killing of M. tuberculosis.

The increased killing of INH associated with cysteine was not due to the amino acid structure of cysteine but to the presence of a thiol group. The notion that a free thiol could boost the activity of bactericidal drugs in M. tuberculosis via increased ROS production is otherwise puzzling since thiols are known radical quenchers and therefore might be considered as antioxidant compounds. In mammalian cells, the combination of vitamin C and vitamin B12 induces an oxidative burst leading to DNA damage (Solovieva et al., 2007). Interestingly, in that work, N-acetylcysteine and reduced glutathione (but not DTT) protected mammalian cells against the cytotoxic effects of the oxidative burst due to the combination of vitamins B12b and C. The authors also noted that DTT had no effect on hydrogen peroxide levels on its own but the combination of vitamins B12b and C and DTT doubled the level of hydrogen peroxide in mammalian cells, This correlates with our observation that NAC was the only free thiol we tested that showed no activity in combination with INH. In this case, the thiol might act as an antioxidant and prevent the necessary oxidative burst. Thus stimulating respiration using the thiol-containing compounds or vitamin C had an important anti-M. tuberculosis synergistic effect when INH or RIF are being used to kill the M. tuberculosis.

The rapid emergence of resistance to INH or RIF in an in vitro culture of M. tuberculosis is rather surprising, since the frequency of spontaneous mutation is about 10⁻⁶ for INH and 10⁻⁸ for RIF. A 5 ml starting culture of 5×10⁶ CFU/ml should contain no pre-existing RIF-resistant mutants and few pre-existing INH-resistant mutants. If the drug-resistant population's growth was due to pre-existing mutants, then it would require about 21 days of doubling to get 10⁷ INH-resistant mutants and no RIF-resistant mutant should arise, but this is not what is observed. Thus, it is possible that the mechanism by which an antibiotic would increase mutational rates via the formation of single-stranded DNA (Rosenberg, 2001) could also happen in M. tuberculosis. The diversity of the katG mutants isolated during clinical INH treatment of M. tuberculosis could indicate that drug treatment accelerates the formation of drug-resistant mutants through increased mutagenesis, as reported in Salmonella, Pseudomonas, Staphylococci, and Mycobacteria. In our study, the increase in ROS production and double-stranded DNA breaks were at their highest in M. tuberculosis treated with INH/Cyst and RIF/Cyst. Therefore, it could be concluded that the INH/Cyst and RIF/Cyst samples might produce more resistant mutants, yet, just the opposite was observed: sterilization of M. tuberculosis cultures as well as a small reduction in frequency of mutation in M. tuberculosis when cysteine was present.

It was also shown that combining cysteine with INH or RIF did not result in the emergence of drug-resistant mutants and ultimately led to sterilization of the cultures. This implies that the persister population which is often associated with treatment failure and emergence of a drug-resistant population in TB-infected patients can also be killed by the combination of cysteine and one or both of INH and RIF. In this study, and the vitamin C study, addition of a single compound (vitamin C) or a combination of compounds (cysteine and INH or RIF) results in sterilization of M. tuberculosis cultures via, increased ROS production. In both instances, this phenomenon could be reversed by oxygen deprivation or iron scavenging to prevent a Fenton reaction. The persister population, which can be observed in INH-treated M. tuberculosis cultures, was totally eradicated by these two conditions. Generating higher ROS levels would lead to persister depletion and ultimately sterilization of M. tuberculosis cultures. Considering the increasing incidence of MDR-and XDR-TB, the addition of nontoxic small molecules to TB chemotherapy that could prevent the emergence of primary drug-resistant TB and eliminate persister population may be of considerable interest and is worthy of further study.

### MATERIALS AND METHODS

### Example 1

Mycobacterial strains and growth conditions: Attenuated strains of M. tuberculosis were constructed by allelic exchange via specialized transduction from the parental strain H37Rv. Null mutants in M. tuberculosis strains H37Rv, mc27000/mc26230 (ΔpanCD, ΔRD-1) (Sambandamurthy et al., 2002), and show identical growth characteristics in standard atmosphere as the parental strain** (unpublished results). For a full list of strains used in this work, see Table 2.

For metabolomic experiments, cells were grown in Middlebrook 7H9 and 7H10 media (Difco, Sparks, Md.) supplemented with Middlebrook OADC Growth Supplement (Difco, Sparks, Md.), and glycerol (5 mg/ml) and tyloxapol (0.5 mg/ml). For antibiotic treatment, OADC was omitted and replaced with NaCl (0.85 mg/ml) dextrose (2 mg/ml), and bovine albumin-fraction V (5 mg/ml). Media was carbon balanced for single carbon source growth curves (see text). For auxotrophic strains, pantothenic acid (100 μg/ml) was added. All cultures were grown in 490 cm² roller bottles (HSR=26), (Corning, N.Y.) with agitation at 100 rpm and 37° C. or in 30 ml inkwell bottles (Nalgene Rochester, N.Y.) for routine culture.

For experiments requiring growth conditions involving low oxygen, cells were incubated in a controlled atmosphere chamber (Coy Laboratory Products, Grass Lake, Mich.), fitted with an AC100 CO₂ controller (Coy Laboratory Products). The internal atmosphere was composed of 4.96% CO₂, 5.99% H₂, and the balance Nitrogen as reported previously (Baughn et al., 2009).

Antibiotics, authentic standards for mass spectroscopy, and uncoupling agents were purchased from Sigma Aldrich, St. Louis, Mo. Fluorescent ROS dyes were purchased from Molecular Probes, Grand Island, N.Y.

For controlled batch culture experiments, DasGip mini bioreactors were employed (DasGip, Jülich, Germany). A 24 hr calibration of oxygen probes and pH probes was performed in vessels containing 200 ml of sterile media prior to inoculation for each experiment.

For membrane potential measurements, the method of Zilberstein (Zilberstein et al., 1984) was used. Briefly, hypoxic and aerobically growing cultures were harvested in log phase (OD=0.6-0.7) or after 12 days of incubation in a controlled atmosphere chamber. For the determination of ΔΨ, In these experiments, cells (1 ml) are taken directly from the fermenter/chemostat and added (quickly) to 5 ml polystyrene (or glass) test-tubes containing [3H]TPP+ (2.4 nM final concentration). After incubation for 5-10 min at 37° C., the reactions were stopped by the adding 2 ml cold 0.1 M LiCl and rapid filtration through 0.45-uM cellulose-acetate filter (Sartorius). Filters were dried and resuspended in scintillation fluid and counts were recorded on a MicroBeta Liquid Scintillation Counter (Perkin Elmer, Waltham, Mass.).

For MKH2/MK HPLC analysis, samples from batch cultures were taken by use of a sampling device to ensure reproducible and quantitative aliquots within 0.5 s. Samples from batch cultures were taken by pipetting within 2 s. Samples (2 ml) were quenched with 6 ml ice-cold 0.2 M HClO₄ in methanol. Petroleum ether (6 ml; 40-60° C.) was then added rapidly to the mixture, and vortexed for 1 mM. After the mixture was centrifuged (900 g, 2 min), the upper petroleum ether phase was removed, transferred to a test tube, and evaporated to dryness under a flow of nitrogen. Another 6 ml petroleum ether was added to the lower phase, and the vortexing and centrifugation steps were repeated. The upper phases were combined. After evaporation to dryness, extracts could be stored for at least 7 days under nitrogen at −20° C. without any detectable auto-oxidation. Immediately before use, the extracted UQ/ubiquinol was resuspended with a glass rod in 80 μl ethanol and analysed in a HPLC system (Pharmacia LKB gradient pump 2249 system, with an LKB 2151 variable-wavelength monitor) containing a reverse-phase Lichrosorb (Chrompack) 10 RP 18 column (4.6 mm i.d., 250 mm length). The column was equilibrated with ethanol:methanol (1:1, v/v), and this mixture was used as the mobile phase. The flow rate was set at 1 ml min−1. Detection of the quinones was performed at 290 nm for UQs, at 248 nm for MKs, and at 270 nm to record all quinones simultaneously. The amounts of all quinones were calculated from the peak areas using UQ10 and MK4 as standards, according to the method applied by Shestopalov et al. (1997). Methanol, ethanol and petroleum ether were of analytical grade.

Metabolomics: For aqueous metabolite pools, hypoxic and aerobically growing cultures were harvested in log phase (OD=0.6-0.7) or after 12 days of incubation in a controlled atmosphere chamber (Coy Laboratory Products, Grass Lake, Mich.) with 0% O₂, 5% CO₂, 10% H2, balance nitrogen. Briefly, 5 ml cell cultures were rapidly quenched in 10 ml 100% MeOH at −20° C. and centrifuged at 4000 rpm for 10 min at −9° C. Cell pellets were resuspended in Extraction solvent containing 40% ACN, 40% MeOH, 20% H₂O and transferred to tubes containing silica beads then agitated 2× in a FastPrep-24 (MP Bio, Solon, Ohio) with 5 min on ice between beatings. Samples were briefly spun and 750 μl of extract was filtered through a sterile 0.22 μM filter, and then frozen at −80° C. until time of analysis. Extracts were made in triplicate. For organic extracts, a method modified from (Layre et. al. 2011) was used. 5 ml cells were centrifuged at 4000 rpm for 10 min. at 0° C. Cell pellets were washed once in 10 ml PBS, resuspended in 1 ml of CH₃OH, transferred to a 13×100 mm glass tube, and added to 2nd chloroform, rotating overnight. The CHCl₃/CH₃OH suspensions were centrifuged 10 min at 3000 rpm and supernatants were removed using a sterile Pasteur pipette into a clean pre-weighed glass vial and dried under nitrogen for 1 h. Vials were weighed after drying to determine dry weight of lipid extracts. Dried lipids were then resuspended in 750 μl of isopropanol/ACN/water (2:1:1, v/v) and frozen until analysis. Extracts were made in triplicate.

For isotope labeling experiments the method of Watanabe et. al. was followed. Hypoxic and aerobically growing cultures were labeled with [1, 4-13C2] L-aspartic acid or [U-13C1] L-aspartic acid. 187.5 μl of 20 mg/ml ¹³C-sources* were added into 1.5 ml of culture in triplicate and incubated for 20 or 24 h at 37° C. Post-incubation, cultures were rapidly quenched in methanol and aqueous metabolites were extracted using the method detailed above. Extracts were frozen at −80° C. until time of analysis.

Analysis was performed using an Acquity UPLC system (Waters, Manchester, UK) coupled with a Synapt G2 quadrupole-time of flight hybrid mass spectrometer. Column eluents were delivered via Electrospray Ionization. UPLC was performed in HILIC mode gradient elution using an Acquity amide column 1.7 μm (2.1×150 mm) using a method previously described (Paglia et al., 2012). The flow rate is 0.5 mL/min with mobile phase A (100% acetonitrile) and mobile phase B (100% water) both containing 0.1% formic acid. The gradient in both positive and negative mode is 0 min, 99% B; 1 min, 99% B; 16 min, 30% B; 17 mm, 30% B; 19 min 99% B; 20 min 99% B. The mass spectrometer was operated in V mode for high sensitivity using a capillary voltage of 2 kV and a cone voltage of 17 V. The desolvation gas flow rate is 500 L/h, and the source and desolvation gas temperature are 120 and 325° C. MS spectra were acquired in centroid mode from m/z 50 to 1,000 using a scan time of 0.5 s. Leucine enkephalin (2 ng/μL) was used as lock mass (m/z 556.2771 and 554.2615 in positive and negative experiments, respectively).

### Example 2

Bacterial strains, plasmids, phages, and media. The M. tuberculosis strains used in this study were obtained from laboratory stocks. The strains were grown in Middlebrook 7H9 medium (Difco, Sparks, Md.) supplemented with 10% (v/v) OADC enrichment (Difco), 0.2% (v/v) glycerol, and 0.05% (v/v) tyloxapol. The solid media used was Middlebrook 7H10 medium (Difco) supplemented with 10% (v/v) OADC enrichment (Difco) and 0.2% (v/v) glycerol. For experiments involving M. tuberculosis H37Rv ΔRD1 ΔpanCD, D-Pantothenic acid hemicalcium salt (25 mg/l) was added to liquid and solid media. All other chemicals were obtained from Sigma (St. Louis, Mo.).

Growth curves. M. tuberculosis cultures were grown at 37° C. to an OD_{600 nm} of 0.7-1.0. The cultures were diluted 1/50, treated with the appropriate chemicals, and incubated at 37° C. with shaking for the duration of the experiment. OD_{600 nm} was recorded and colony-forming units (CFUs) were obtained by plating serial dilutions (see media above) and incubating the plates at 37° C. for 4 weeks.

Viability under anaerobic conditions. M. tuberculosis H37Rv ΔRD1 ΔpanCD was grown under a normal aerobic atmosphere to OD₆₀₀ 0.5-0.8 and diluted 1/50 before being shifted to an anaerobic chamber (<0.0001% O₂, 5% CO₂, 10% H₂, 85% N₂). After 24 h of anaerobic incubation, the culture was divided and treated with RIF, cysteine and the combination. At each time point, aliquots were taken and removed from the chamber to be serial-diluted and plated. The plates were incubated at 37° C. under aerobic conditions for 4 weeks.

Flow cytometry analysis of ROS. M. tuberculosis H37Rv ΔRD1 Δpan/CD was grown to O.D. 1 and diluted to O.D. 0.2 in fresh media containing antibiotic. Aliquots were taken at the indicated time points and cells were washed twice in MP Buffer and stained with dihydroethidium for 30 min at 37° C. Cells were immediately analyzed on a BD FACS Calibur (BD Biosciences, San Jose, Calif.) with the following instrument settings: forward scatter, E01 log gain; side scatter, 474V log; fluorescence (FL1), 674V log; fluorescence (FL2), 613V log; and threshold value, 52. For each sample, 100,000 events were acquired, and analysis was done by gating intact cells using log phase controls.

TUNEL staining for DNA breaks. Terminal deoxynucleotidyltransferase-mediated dUTP-biotin nick end labeling was used to detect DNA breaks in mycobacteria undergoing antibiotic treatment. Aliquots of mycobacterial cells, coming from the same cells as the ones used for ROS analysis, were removed at indicated time points and washed once in PBS, then fixed in 2% paraformaldehyde (in PBS) for 30 mm at room temperature, whereupon cells were centrifuged and the fixing solution was removed. The pellets were permeabilized for 2 min on ice, rewashed in PBS, then resuspended in 100 μl of TUNEL reaction mix from the in situ cell death detection kit (Roche Molecular Biochemicals, Indianapolis, Ind.), and incubated at 37° C. in the dark for 1 hr. The reaction mixture was then washed in PBS once and saved at 4° C. until analysis. Analysis was done by flow cytometry using the instrument settings described above, 100,000 events were acquired per sample. Analysis was done by gating intact cells using cells were incubated with label solution only (no terminal transferase) as negative control. Cells positive for TUNEL staining were quantified as percentages of total gated cells.

Microarray Analysis. Triplicate samples were prepared by adjusting an exponentially growing culture of M. tuberculosis H37Rv to an OD_{600 nm} of 0.2 in a 50 ml volume in a 500 ml roller bottle and treating with INH (7.3 μM), both with and without cysteine (4 mM). After 4 days of incubation rolling at 37° C., cultures were harvested. Extraction of RNA, preparation of cDNA, and microarray analysis were performed, as described previously (Vilcheze et al., 2010). The array data have been deposited in the Gene Expression Omnibus at NCB1 (www.ncbi.nlm.nih.gov/geo).

Quantification of ferrous ion concentrations. M. tuberculosis H37Rv ΔRD1 ΔpanCD was grown to an OD_{600 nm} ≈0.8, diluted 1/10, and incubated with INH, cysteine, RIF or the combination at the indicated concentrations at 37° C. while shaking for three days. Extraction and quantification of iron was done as previously described (Riemer et al., 2004). Briefly, the cultures were centrifuged and the cell pellets were washed twice with ice cold PBS, resuspended. in 1 mL 50 mM NaOH with glass beads and lysed using a Fast Prep machine. To quantify ferrous ion concentrations, the lysate (0.1 mL) was mixed with 10 mM HCl (0.1 mL) and incubated at 60° C. for two hours. The samples were returned to room temperature before adding the iron detection reagent (6.5 mM ferrozine+6.5 mM neocuproine+2.5 M ammonium acetate; 0.03 ml). The sample absorbance was read at 550 nm, The iron concentrations were determined based on a standard curve obtained with increasing concentrations of ferric chloride and normalized to protein content.

Determination of total thiol concentration. M. tuberculosis H37Rv ΔRD1 ΔpanCD was grown to an OD_{600 nm}≈0.3, treated with the appropriate chemicals, and incubated for 24 h at 37° C. while shaking. The cultures (10 ml) were spun down and the cell pellets were washed once with PBS and resuspended in 0.5 ml PBS. Glass beads were added and the cell pellets were lysed using the Thermo Scientific FastPrep machine (45 sec, speed 6, 3 times). After cooling, the samples were centrifuged and the supernatants were filter-sterilized before analysis. The total thiol concentration was Obtained using Ellman's reagent. Briefly, a 1 ml solution containing 50 mM Tris (pH 8.0), 5 mM 5,5′-dithiobis(2-nitrobenzoic acid) (10 μl), and the lysate to quantify was measured spectrophotometrically at 412 nm using the following extinction coefficient: ε_{412 nm} 2-nitro-5-thiobenzoate anion 14,150 M⁻¹ cm⁻¹.

Determination of cystine level by UPLC-MS. M. tuberculosis H37Rv ΔRD1 ΔpanCD was grown to an OD_{600 min}≈0.3, treated with the appropriate chemicals, and incubated for 24 h at 37° C. while shaking. The cultures (5 ml) were quenched with 10 ml of cold methanol and spun down at 0° C. for 10 min. The cell pellets were resuspended in 1 ml acetonitrile/methanol/water (40/40/20; v/v/v). Glass beads (0.1 ml) were added and lysed using the Thermo Scientific FastPrep machine (45 sec, speed 6, 3 times). After cooling, the samples were centrifuged for 1 min and the supernatants were filter-sterilized. The samples were analyzed on an Acquity UPLC (Waters, Manchester, UK) coupled with a quadrupole-time of flight mass spectrometer (Synapt G12, Waters). The UPLC column was Acquity amide column 1.7 μm. The flow rate was 0.5 mL/min, the column temperature was set at 55° C. The mobile phases were: A, acetonitrile with 0.1% formic acid; B, water with 0.1% formic acid. The conditions used for the UPLC were: 0 min, 99% A; 1 min, 99% A; 14 min, 65% A; 17 min, 40% A; 18 min, 40% A; 19 min, 99% A; 20 min, 99% A. The MS conditions were: acquisition mode, MS^{E}; ionization mode, ESI (+/−) capillary voltage, 2 kV (positive), 3 kv (negative); cone voltage, 30 V; desolvation gas flow, 800 L/h; desolvation temperature, 550° C.; source temperature. 120° C., acquisition range, 50 to 1200 m/z with a scan time of 0.5 s. Leucine enkephalin (2 ng/μL) was used as lock mass. Data generated from the experiment were processed and analyzed with Waters MarkerLynx software, which integrates, normalizes, aligns MS data points and converts them into exact mass retention time pairs to build a matrix composed of retention time, exact mass m/z, and intensity pairs. Cysteine and cystine standards were run to determine retention time and m/z.

### REFERENCES

1. Allison, K. R., Brynildsen, M. P., and Collins, J. J. (2011). Metabolite-enabled eradication of bacterial persisters by aminoglycosides. Nature 473, 216-220.
Andries, K., Verhasselt, P., Guillemont, J., Göhlmann, Neefs, J.-M., Winkler, H., Van Gestel, J., Timmerman, P., Zhu, M., Lee, E., et al. (2005). A diarylquinoline drug active on the ATP synthase of Mycobacterium tuberculosis. Science (New York, N.Y.) 307, 223-227.
3. Balaban, N. Q., Merrin, J., Chait, R., Kowalik, L., and Leibler, S. (2004). Bacterial persistence as a phenotypic switch. Science (New York, N.Y.) 305, 1622-1625.
4. Bardarov, S., Bardarov Jr, S., Pavelka Jr, M. S., Sambandamurthy, V., Larsen, M., Tufariello, J., Chan, J., Hatfull, G., and Jacobs Jr, W. R. (2002). Specialized transduction: an efficient method for generating marked and unmarked targeted gene disruptions in Mycobacterium tuberculosis, M. bovis BCG and M. smegmatis. Microbiology (Reading, England) 148, 3007-3017.
5. Barry, C. E., Boshoff, H. L., Dartois, V., Dick, T., Ehrt, S., Flynn, J., Schnappinger, D., Wilkinson, R. J., and Young, D. (2009). The spectrum of latent tuberculosis: rethinking the biology and intervention strategies, Nature Reviews. Microbiology 7, 845-855.
6. Baughn, A. D., Garforth, S. J., Vilchèze, C., and Jacobs, W. R. (2009). An anaerobic-type alpha-ketoglutarate ferredoxin oxidoreductase completes the oxidative tricarboxylic acid cycle of Mycobacterium tuberculosis. PLoS Pathogens 5, e1000662.
7. Bavesh D, Kana, Machowski, E. E., Schechter, N., Teh, J.-S., Rubin, H., Mizrahi, V., and Drancourt, M. (2009). Mycobacterium: Genomics and Molecular Biology (Caister Academic Press).
8. Bekker, M., Kramer, G., Hartog, A. F., Wagner, M. J., De Koster, C. G., Hellingwerf, K. J., and De Mattos, M. J. T. (2007). Changes in the redox state and composition of the quinone pool of Escherichia coli during aerobic batch-culture growth. Microbiology (Reading, England) 153, 1974-1980.
9. Berney, M., and Cook, G. M. (2010). Unique flexibility in energy metabolism allows mycobacteria to combat starvation and hypoxia. PloS One 5, e8614.
10. Beste, D. J. V, Espasa, M., Bonde, B., Kierzek, A. M., Stewart, G. R., and McFadden, J. (2009). The genetic requirements for fast and slow growth in mycobacteria. PloS One 4, e5349.
11. Bigger, J. J. (1944). Treatment of staphylococcal infections with penicillin by intermittent sterilisation. The Lancet 247, 497-500.
12. Borisov, V. B., Murali, R., Verkhovskaya, M. L., Bloch, D. a, Han, H., Gennis, R. B., and Verkhovsky, M. I. (2011). Aerobic respiratory chain of Escherichia coli is not allowed to work in fully uncoupled mode. Proceedings of the National Academy of Sciences of the United States of America. 108, 17320-17324.
13. De Carvalho, L. P. S., Fischer, S. M., Marrero, J., Nathan, C., Ehrt, S., and Rhee, K. Y. (2010). Metabolomics of Mycobacterium tuberculosis reveals compartmentalized co-catabolism of carbon substrates. Chemistry & Biology 17, 1122-1131.
14. Cecchini, G., Schröder, I., Gunsalus, R. P., and Maklashina, E. (2002). Succinate dehydrogenase and fumarate reductase from Escherichia coli. Biochimica Et Biophysica Acta. 1553, 140-157.
15. Cox, R., and Cook, G. (2007), Growth regulation in the mycobacterial cell. Current Molecular Medicine 231-245.
16. Das, B., Kashino, S. S., Pulu, I., Kalita, D., Swami, V., Yeger, H., Felsher, D. W., and Campos-Neto, A. (2013). CD271+ Bone Marrow Mesenchymal Stem Cells May Provide a Niche for Dormant Mycobacterium tuberculosis, Science Translational Medicine 5, 170ra13.
17. Dhar, N., and McKinney, J. D. (2007). Microbial phenotypic heterogeneity and antibiotic tolerance. Current Opinion in Microbiology 10, 30-38.
18. Diacon, A., and Pym, A. (2009). The diarylquinoline TMC207 for multidrug-resistant tuberculosis, . . . England Journal of . . . 2397-2405.
19. Dörr, T., Vulić, M., and Lewis, K. (2010). Ciprofloxacin causes persister formation by inducing the TisB toxin in Escherichia coli. PLoS Biology 8, e1000317.
20. Dry, I., Moore, A., Day, D., and Wiskich, J. (1989). Regulation of alternative pathway activity in plant mitochondria: nonlinear relationship between electron flux and the redox poise of the quinone pool. Archives of Biochemistry and . . . 273.
21. Ehrt, S., Guo, X. V, Hickey, C. M., Ryou, M., Monteleone, M., Riley, L. W., and Schnappinger, D. (2005). Controlling gene expression in mycobacteria with anhydrotetracycline and Tet repressor. Nucleic Acids Research 33, e21.
22. Filippini, P., Iona, E., Piccaro, G., Peyron, P., Neyrolles, O., and Fattorini, L. (2010). Activity of drug combinations against dormant Mycobacterium tuberculosis. Antimicrobial Agents and Chemotherapy 54, 2712-2715.
23. Gomez, J. E., and McKinney, J. D. (2004). M. tuberculosis persistence, latency, and drug tolerance. Tuberculosis 84, 29-44.
24. Grant, S. S., Kaufmann, B. B., Chand, N. S., Haseley, N., Hung, D. T., and Schmidt Grant, S. (2012). Eradication of bacterial persisters with antibiotic-generated hydroxyl radicals. Proceedings of the National Academy of Sciences of the United States of America 109, 12147-12152.
25. Hunte, C., Palsdottir, H., and Trumpower, B. L. (2003). Protonmotive pathways and mechanisms in the cytochrome bc1 complex. FEBS Letters 545, 39-46.
26. Jasmer, R. M., Bozeman, L., Schwartzman, K., Cave, M. D., Saukkonen, J. J., Metchock, B., Khan, A., and Burman, W. J. (2004). Recurrent tuberculosis in the United States and Canada: relapse or reinfection? American Journal of Respiratory and Critical Care Medicine 170, 1360-1366.
27. Jayaram, R., Gaonkar, S., and Kaur, P. (2003). Pharmacokinetics-pharmacodynamics of rifampin in an aerosol infection model of tuberculosis. Antimicrobial Agents . . . 47, 2118-2124.
28. Jayaram, R., and Shandil, R. (2004). isoniazid pharmacokinetics-pharmacodynamics in an aerosol infection model of tuberculosis. Antimicrobial Agents . . . 48, 2951-2957.
29. Juhnke, H. D., Hiltscher, H., Nasiri, H. R., Schwalbe, H., and Lancaster, C. R. D. (2009). Production, characterization and determination of the real catalytic properties of the putative “succinate dehydrogenase” from Wolinella succinogenes. Molecular Microbiology 71, 1088-1101.
30. Keren, I., Kaldalu, N., Spoering, A., Wang, Y., and Lewis, K. (2004). Persister cells and tolerance to antimicrobials. FEMS Microbiology Letters 230, 13-18.
31. Kint, C. I., Verstraeten, N., Fauvart, M., and Michiels, J. (2012). New-found fundamentals of bacterial persistence. Trends in Microbiology 20, 577-585.
32. Kohanski, M. A., Dwyer, D. J., Hayete, B., Lawrence, C. a, and Collins, J. J. (2007). A common mechanism of cellular death induced by bactericidal antibiotics. Cell 130, 797-810.
33. Koul, A., Vranckx, L., Dendouga, N., Balemans, W., Van den Wyngaert, I., Vergauwen, K., Göhlmann, H. W. H., Willebrords, R., Poncelet, A., Guillemont, J., et al. (2008). Diarylquinolines are bactericidal for dormant mycobacteria as a result of disturbed ATP homeostasis. The Journal of Biological Chemistry 283, 25273-25280.
34. Kröger, A., Biel, S., Simon, J., Gross, R., Unden, G., and Lancaster, C. R. D. (2002). Fumarate respiration of Wolinella succinogenes: enzymology, energetics and coupling mechanism. Biochimica Et Biophysica Acta 1553, 23-38.
35. Kröger, A., and Klingenberg, M. (1973). The kinetics of the redox reactions of ubiquinone related to the electron-transport activity in the respiratory chain. European Journal of Biochemistry/FEBS 34, 358-368.
36. Kumar, A., Farhana, A., Guidry, L., Saini, V., Hondalus, M., and Steyn, A. J. C. (2011). Redox homeostasis in mycobacteria: the key to tuberculosis control? Expert Reviews in Molecular Medicine 13, e39.
37. Lemos, R. S., Fernandes, A. S., Pereira, M. M., Gomes, C. M., and Teixeira, M. (2002). Quinol:fumarate oxidoreductases and succinate:quinone oxidoreductases: phylogenetic relationships, metal centres and membrane attachment. Biochimica Et Biophysica Acta 1553, 158-170.
38. Lewis, K. (2010). Persister cells. Annual Review of Microbiology 64, 357-372.
39. Li, Y., and Zhang, Y. (2007). PhoU is a persistence switch involved in persister formation and tolerance to multiple antibiotics and stresses in Escherichia coli. Antimicrobial Agents and Chemotherapy 51, 2092-2099.
40. Loebel, R. O., Shorr, E., and Richardson, H. B. (1933). The Influence of Adverse Conditions upon the Respiratory Metabolism and Growth of Human Tubercle Bacilli. Journal of Bacteriology 26, 167-200.
41. Ma, C., Sim, S., Shi, W., Du, L., Xing, D., and Zhang, Y. (2010). Energy production genes sucB and ubiF are involved in persister survival and tolerance to multiple antibiotics and stresses in Escherichia coli. FEMS Microbiology Letters 303, 33-40.
42. McKinney, J. D., Höner zu Bentrup, K., Muñoz-Elías, E. J., Miczak, a, Chen, B., Chan, W. T., Swenson, D., Sacchettini, J. C., Jacobs, W. R., and Russell, D. G. (2000). Persistence of Mycobacterium tuberculosis in macrophages and mice requires the glyoxylate shunt enzyme isocitrate lyase. Nature 406, 735-738.
43. Megehee, J. a, Hosler, J. P., and Lundrigan, M. D. (2006). Evidence for a cytochrome bcc-aa3 interaction in the respiratory chain of Mycobacterium smegmatis. Microbiology (Reading, England) 152, 823-829.
44. Mitchell, P. (1975). Protonmotive redox mechanism of the cytochrome b-c1 complex in the respiratory chain: protonmotive ubiquinone cycle, FEBS Letters 56, 1-6.
45. Narayanan, S., Swaminathan, S., Supply, P., Shanmugam, S., Narendran, G., Hari, L., Ramachandran, R., Locht, C., Jawahar, M. S., and Narayanan, P. R. (2010). Impact of HIV infection on the recurrence of tuberculosis in South India. The Journal of Infectious Diseases 201, 691-703.
46. Neyrolles, O., Hernández-Pando, R., Pietri-Rouxel, F., Fornès, P., Tailleux, L., Barrios Payan, J. A., Pivert, E., Bordat, Y., Aguilar, D., Prévost, M.-C., et al. (2006). Is adipose tissue a place for Mycobacterium tuberculosis persistence? PloS One 1, e43.
47. Noble, M., and Smith, J. (2003). Redox state as a central modulator of precursor cell function. Annals of the New . . . .
48. Ogasawara, M., and Zhang, H. (2009). Redox regulation and its emerging roles in stem cells and stem-like cancer cells. Antioxidants & Redox Signaling 11.
49. Paglia, G., Hrafnsdóttir, S., Magnúsdóttir, M., Fleming, R. M. T., Thorlacius, S., Palsson, B. Ø., and Thiele, I. (2012). Monitoring metabolites consumption and secretion in cultured cells using ultra-performance liquid chromatography quadrupole-time of flight mass spectrometry (UPLC-Q-ToF-MS). Analytical and Bioanalytical Chemistry 402, 1183-1198.
50. Piddock, L. J. V. (2000). Accumulation of rifampicin by Mycobacterium aurum, Mycobacterium smegmatis and Mycobacterium tuberculosis. Journal of Antimicrobial Chemotherapy 45, 159-165.
51. Roos, D., Eckmann, C. M., Yazdanbakhsh, M., Hamers, M. N., and De, M. (1984). Excretion of Superoxide by Phagocytes Measured with Cytochrome c entrapped in. Journal of Biological Chemistry 1770-1775.
52. Sacchettini, J. C., Rubin, E. J., and Freundlich, J. S. (2008). Drugs versus bugs: in pursuit of the persistent predator Mycobacterium tuberculosis. Nature Reviews. Microbiology 6, 41-52.
53. Sambandamurthy, V. K., Wang, X., Chen, B., Russell, R. G., Derrick, S., Collins, F. M., Morris, S. L., and Jacobs, W. R. (2002). A pantothenate auxotroph of Mycobacterium tuberculosis is highly attenuated and protects mice against tuberculosis. Nature Medicine 8, 1171-1174.
54. Sassetti, C. M., Boyd, D. H., and Rubin, E. J. (2001). Comprehensive identification of conditionally essential genes in mycobacteria. Proceedings of the National Academy of Sciences of the United States of America 98, 12712-12717.
55. Sassetti, C. M., and Rubin, E. J. (2003). Genetic requirements for mycobacterial survival during infection. Proceedings of the National Academy of Sciences of the United States of America 100, 12989-12994.
56. Shah, N. S., Richardson, J., Moodley, P., Moodley, S., Babaria, P., Ramtahal, M., Heysell, S. K., Li, X., Moll, A. P., Friedland, G., et al. (2011). Increasing drug resistance in extensively drug-resistant tuberculosis, South Africa. Emerging Infectious Diseases 17, 510-513.
57. Shapiro, H. M. (2008). Flow cytometry of bacterial membrane potential and permeability. Methods in Molecular Medicine 142, 175-186.
58. Shi, L., Sohaskey, C. D., Kana, B. D., Dawes, S., North, R. J., Mizrahi, V., and Gennaro, M. L. (2005). Changes in energy metabolism of Mycobacterium tuberculosis in mouse lung and under in vitro conditions affecting aerobic respiration. Proceedings of the National Academy of Sciences of the United States of America 102, 15629-15634.
59. Smith, L., Davies, H., and Nava, M. (1974). Oxidation and reduction of soluble cytochrome c by membrane-bound oxidase and reductase systems. Journal of Biological Chemistry 2904-2910.
60. Spoering, A. L., Vulic, M., and Lewis, K. (2006). GlpD and PlsB participate in persister cell formation in Escherichia coli. Journal of Bacteriology 188, 5136-5144.
61. Udwadia, Z., and Amale, R. (2012). Totally drug-resistant tuberculosis in India. Clinical Infectious . . . 54, 579-581.
62. Unoson, C., and Wagner, E. G. H. (2008). A small SOS-induced toxin is targeted against the inner membrane in Escherichia cob. Molecular Microbiology 70, 258-270.
63. Vega, N. M., Allison, K. R., Khalil, A. S., and Collins, J. J. (2012). Signaling-mediated bacterial persister formation. Nature Chemical Biology 8, 431-433.
64. Vilchèze, C., and Jacobs, W. R. (2007). The Mechanism of Isoniazid Killing: Clarity Through the Scope of Genetics. Annual Review of Microbiology 61, 35-50.
65. Voskuil, M. I. (2004). Mycobacterium tuberculosis gene expression during environmental conditions associated with latency. Tuberculosis (Edinburgh, Scotland) 84, 138-143.
66. Wallis, R. S., Patil, S., Cheon, S. H., Edmonds, K., Phillips, M., Perkins, M. D., Joloba, M., Namale, a, Johnson, J, L., Teixeira, L., et al. (1999). Drug tolerance in Mycobacterium tuberculosis. Antimicrobial Agents and Chemotherapy 43, 2600-2606.
67. Wang, Q., Zhang, Y., Yang, C., and Xiong, H. (2010). Acetylation of metabolic enzymes coordinates carbon source utilization and metabolic flux. Science . . . 327.
68. Watanabe, S., Zimmermann, M., Goodwin, M. B., Sauer, U., Barry, C. E., and Boshoff, H. I. (2011). Fumarate Reductase Activity Maintains an Energized Membrane in Anaerobic Mycobacterium tuberculosis. PLoS Pathogens 7, e1002287.
69. Wayne, L. G., and Hayes, L. G., (1996). An in vitro model for sequential study of shiftdown of Mycobacterium tuberculosis through two stages of nonreplicating persistence. Infection and Immunity 64, 2062-2069.
70. WHO (2012a). GLOBAL TUBERCULOSIS REPORT.
71. WHO (2012b). Treatment of tuberculosis guidelines (Geneva, Switzerland).
72. Winder, F., and Collins, P. (1970). Inhibition by isoniazid of synthesis of mycolic acids in Mycobacterium tuberculosis, Journal of General Microbiology 41-48.
73. Wiuff, C., Zappala, R. M., Regoes, R. R., Garner, K. N., Baquero, F., and Levin, B. R. (2005), Phenotypic Tolerance: Antibiotic Enrichment of Noninherited Resistance in Bacterial Populations. Society 49, 1483-1494.
74. Zhang, Z., Huang, L., Shulmeister, V. M., Chi, Y. I., Kim, K. K., Hung, L. W., Crofts, A. R., Berry, E. A., and Kim, S. H. (1998). Electron transfer by domain movement in cytochrome bc1. Nature 392, 677-684.
75. Zilberstein, D., Agmon, V., Schuldiner, S., and Padan, E. (1984). Escherichia coli intracellular pH, membrane potential, and cell growth. Journal of Bacteriology 158, 246-252.
76. Park, S. & J. A., Imlay, (2003) High levels of intracellular cysteine promote oxidative DNA damage by driving the fenton reaction. J. Bacteriol. 185: 1942-1950.
77. Piccaro, G., F. Giannoni, P. Filippini, A. Mustazzolu & L. Fattorini, (2013) Activities of drug combinations against Mycobacterium tuberculosis grown in aerobic and hypoxic acidic conditions. Antimicrob. Agents Chemother. 57: 1428-1433
78. Roy, M. K., Y. Kuwahara, K. Hara, Y. Watanabe & Y. Tamai, (2002) Antimutagenic effect of amino acids on the mutagenicity of N-methyl-N′-nitro-N-nitrosoguanidine (MNNG). Biosci. Biotechnol. Biochem. 66: 1400-1402.
79. Solovieva, M. E., V. V. Soloviev & V. S. Akatov, (2007) Vitamin B12b increases the cytotoxicity of short-time exposure to ascorbic acid, inducing oxidative burst and iron-dependent DNA damage. Eur. J. Pharmacol. 566: 206-214.
80. Vilchèze, C., F, Wang, M. Arai, M. H. Hazbon, R. Colangeli, L. Kremer, T. R. Weisbrod, D. Alland, J. C. Sacchettini & W. R. Jacobs, Jr., (2006) Transfer of a point mutation in Mycobacterium tuberculosis inhA resolves the target of isoniazid. Nat. Med. 12: 1027-1029.
81. Vilchèze, C., T. Hartman, B. Weinrick & W. R. Jacobs, Jr., (2013) Mycobacterium tuberculosis is extraordinarily sensitive to killing by a vitamin C-induced Fenton reaction. Nat. Commun. 4: 1881.
82. Ward, S. K., E. A. Hoye & A. M. Talaat, (2008) The global responses of Mycobacterium tuberculosis to physiological levels of copper. J. Bacteriol. 190: 2939-2946.
83. Wardman, P. & L. P. Candeias, (1996) Fenton chemistry: an introduction. Radiat. Res. 145: 523-531.

1. A method for identifying an agent as an enhancer of an anti-tuberculosis medication, or as a combination agent for treating resistant and/or dormant Mycobacteria tuberculosis infection in a subject, comprising:
quantifying activity of an amount of Mycobacteria tuberculosis succinate dehydrogenase I under conditions permitting succinate dehydrogenase I activity,
contacting the Mycobacteria tuberculosis succinate dehydrogenase I with the agent and quantifying activity of the amount of Mycobacteria tuberculosis succinate dehydrogenase I in the presence of the agent,
comparing the quantified amounts,
and identifying the agent as an enhancer, or not, of an anti-tuberculosis medication, or as a combination agent, or not, for treating resistant and/or dormant Mycobacteria tuberculosis infection,
wherein a decreased activity of Mycobacteria tuberculosis succinate dehydrogenase I in the presence of the agent as compared to in the absence of the agent indicates that the agent is an enhancer of an anti-tuberculosis medication, or is a combination agent for treating resistant and/or dormant Mycobacteria tuberculosis infection.

2. (canceled)

3. A method of enhancing efficacy of an anti-tuberculosis medication in treating tuberculosis in a subject or in treating resistant and/or dormant Mycobacteria tuberculosis infection in a subject, comprising:
administering to the subject who has, is or will be receiving the anti-tuberculosis medication an amount of an enhancer of Mycobacteria tuberculosis respiration effective to enhance the efficacy of an anti-tuberculosis medication in treating tuberculosis or resistant and/or dormant Mycobacteria tuberculosis infection.

4. A method of treating tuberculosis in a subject, or of treating resistant and/or dormant Mycobacteria tuberculosis infection in a subject, comprising administering to the subject an amount of an anti-tuberculosis antibiotic medication and an amount of an enhancer of Mycobacteria tuberculosis respiration effective to treat tuberculosis in a subject, or effective to treat resistant and/or dormant Mycobacteria tuberculosis infection in a subject.

5. The method of claim 3, wherein the anti-tuberculosis medication comprises one or more of isoniazid, rifampicin, pyrazinamide, ethambutol, a fluoroquinolone, amikacin, capreomycin or kanamycin.

6. The method of claim 3, wherein the anti-tuberculosis medication comprises rifampicin and isoniazid.

7. The method of claim 3, wherein the agent or enhancer of respiration is an inhibitor of Mycobacteria tuberculosis succinate dehydrogenase I.

8. The method of claim 3, wherein the agent or enhancer of respiration is a small organic molecule of 2000 daltons or less, an aptamer, an RNAi molecule, a peptide, a fusion protein, an antibody, or a fragment of an antibody.

9. The method of claim 3, wherein the enhancer of respiration inhibits expression of Mycobacteria tuberculosis succinate dehydrogenase I or inhibits activity of Mycobacteria tuberculosis succinate dehydrogenase I.

10. The method of claim 3, wherein the Mycobacteria tuberculosis succinate dehydrogenase I is encoded by an operon comprising Mycobacteria tuberculosis genes Rv0247c, Rv0248c, and Rv0249c.

11. The method of claim 3, wherein the agent or enhancer of respiration comprises a free thiol group or is vitamin C.

12. The method of claim 11, wherein the agent or enhancer of respiration comprises a free thiol group and is a cysteine, dithiothreitol or penicillamine.

13. The method of claim 11, wherein the anti-tuberculosis medication comprises isoniazid.

14. The method of claim 11, wherein the anti-tuberculosis medication comprises rifampicin.

15. The method of claim 3, wherein the tuberculosis is multidrug-resistant (MDR).

16. The method of claim 3, wherein the tuberculosis is extensively drug-resistant (XDR).

17-27. (canceled)
